SEQUENCE LISTING (71 sequences)

SEQ ID NO: 1 - DNA, 430 bp, Mycobacterium tuberculosis
  fragment of Mycobacterium tuberculosis IS6110 sequence, Sequence 1
gagacctcagccggcggctggtctctggcgttgagcgtagtaggcagcctcgagttcgaccggcgggacgtcgccgcagtactggtagaggcggcgatggttgaaccagtcgacccagcgcgcggtggccaactcgacatcctcgatggaccgccagggcttgccgggtttgatcagctcggtcttgtataggccgttgatcgtctcggctagtgcattgtcataggagcttccgaccgctccgaccgacggttggatgcctgcctcggcgagccgctcgctgaaccggatcgatgtgtactgagatcccctatccgtatggtggataacgtctttcaggtcgagtacgccttcttgttggcgggtccagatggcttgctcgatcgcgtcgaggaccatggaggtggccatcgtggaagcgacccgccag

SEQ ID NO: 2 - DNA, 38 bp, Artificial Sequence
  Description of Artificial Sequencesynthetic Forward amplification primer oligonucleotide, Sequence 2
gcattatagtacctgtctcctcagcactgagatcccct

SEQ ID NO: 3 - DNA, 37 bp, Artificial Sequence
  Description of Artificial Sequencesynthetic Reverse amplification primer oligonucleotide, Sequence 3
ttgaatagtcggttacttcctcagcgcgtactcgacc

SEQ ID NO: 4 - DNA, 13 bp, Artificial Sequence
  Description of Artificial Sequencesynthetic Forward bumper primer, Sequence 4
cgctgaaccggat

SEQ ID NO: 5 - DNA, 13 bp, Artificial Sequence
  Description of Artificial Sequencesynthetic Reverse bumper primer, Sequence 5
tggacccgccaac

SEQ ID NO: 6 - DNA, 11 bp, Artificial Sequence
  Description of Artificial Sequencesynthetic Probe sequence for endonuclease IV assay
tccgtntggtg

SEQ ID NO: 7 - DNA, 41 bp, Artificial Sequence
  Description of Artificial Sequencesynthetic target amplified by isothermal strand displacement method coupled to endonuclease IV signal amplification method
agtcacagtcggtgccaatgtggcgggcaagggccgagtcg

SEQ ID NO: 8 - DNA, 10 bp, Artificial Sequence
  Description of Artificial Sequencesynthetic Fluorescence Resonance Energy Transfer (FRET) probe with quencher at 5′-end and fluorescent dye coupled to 3′-hydroxyl through phosphate bond and rigid linker
ctcggccctt

SEQ ID NO: 9 - DNA, 11 bp, Artificial Sequence
  Description of Artificial Sequencesynthetic short enhancer oligonucleotide
cccgccacatt

SEQ ID NO: 10 - DNA, 10 bp, Artificial Sequence
  Description of Artificial SequenceN.BstNBI nicking enzyme specificity site
gagtcnnnnn

SEQ ID NO: 11 - DNA, 10 bp, Artificial Sequence
  Description of Artificial SequenceN.AlwI nicking enzyme specificity site
ggatcnnnnn

SEQ ID NO: 12 - DNA, 10 bp, Artificial Sequence
  Description of Artificial SequenceN.Bst9I nicking enzyme specificity site
gagtcnnnnn

SEQ ID NO: 13 - DNA, 11 bp, Artificial Sequence
  Description of Artificial SequenceN.MlyI nicking enzyme specificity site
gagtcnnnnnn

SEQ ID NO: 14 - DNA, 12 bp, Artificial Sequence
  Description of Artificial SequenceNb.SapI-1 (variant 33) nicking enzyme specificity site
gctcttcnnnnn

SEQ ID NO: 15 - DNA, 11 bp, Artificial Sequence
  Description of Artificial SequenceAarI type IIS palindromic restriction endonuclease recognition sequence
cacctgcnnnn

SEQ ID NO: 16 - DNA, 15 bp, Artificial Sequence
  Description of Artificial SequenceAarI type IIS palindromic restriction endonuclease recognition sequence complement
nnnnnnnngcaggtg

SEQ ID NO: 17 - DNA, 13 bp, Artificial Sequence
  Description of Artificial SequenceAceIII type IIS palindromic restriction endonuclease recognition sequence
cagctcnnnnnnn

SEQ ID NO: 18 - DNA, 17 bp, Artificial Sequence
  Description of Artificial SequenceAceIII type IIS palindromic restriction endonuclease recognition sequence complement
nnnnnnnnnnngagctg

SEQ ID NO: 19 - DNA, 22 bp, Artificial Sequence
  Description of Artificial SequenceAcuI type IIS palindromic restriction endonuclease recognition sequence
ctgaagnnnnnnnnnnnnnnnn

SEQ ID NO: 20 - DNA, 20 bp, Artificial Sequence
  Description of Artificial SequenceAcuI type IIS palindromic restriction endonuclease recognition sequence complement
nnnnnnnnnnnnnncttcag

SEQ ID NO: 21 - DNA, 10 bp, Artificial Sequence
  Description of Artificial SequenceAlwI type IIS palindromic restriction endonuclease recognition sequence complement
nnnnngatcc

SEQ ID NO: 22 - DNA, 13 bp, Artificial Sequence
  Description of Artificial SequenceBbr7I type IIS palindromic restriction endonuclease recognition sequence
gaagacnnnnnnn

SEQ ID NO: 23 - DNA, 17 bp, Artificial Sequence
  Description of Artificial SequenceBbr7I type IIS palindromic restriction endonuclease recognition sequence complement
nnnnnnnnnnngtcttc

SEQ ID NO: 24 - DNA, 12 bp, Artificial Sequence
  Description of Artificial SequenceBbsI type IIS palindromic restriction endonuclease recognition sequence complement
nnnnnngtcttc

SEQ ID NO: 25 - DNA, 13 bp, Artificial Sequence
  Description of Artificial SequenceBbyI type IIS palindromic restriction endonuclease recognition sequence
gcagcnnnnnnnn

SEQ ID NO: 26 - DNA, 17 bp, Artificial Sequence
  Description of Artificial SequenceBbyI type IIS palindromic restriction endonuclease recognition sequence complement
nnnnnnnnnnnngctgc

SEQ ID NO: 27 - DNA, 10 bp, Artificial Sequence
  Description of Artificial SequenceBccI type IIS palindromic restriction endonuclease recognition sequence complement
nnnnngatgg

SEQ ID NO: 28 - DNA, 12 bp, Artificial Sequence
  Description of Artificial SequenceBciVI type IIS palindromic restriction endonuclease recognition sequence
gtatccnnnnnn

SEQ ID NO: 29 - DNA, 11 bp, Artificial Sequence
  Description of Artificial SequenceBciVI type IIS palindromic restriction endonuclease recognition sequence complement
nnnnnggatac

SEQ ID NO: 30 - DNA, 10 bp, Artificial Sequence
  Description of Artificial SequenceBfuAI type IIS palindromic restriction endonuclease recognition sequence
acctgcnnnn

SEQ ID NO: 31 - DNA, 14 bp, Artificial Sequence
  Description of Artificial SequenceBfuAI type IIS palindromic restriction endonuclease recognition sequence complement
nnnnnnnngcaggt

SEQ ID NO: 32 - DNA, 11 bp, Artificial Sequence
  Description of Artificial SequenceBmrI type IIS palindromic restriction endonuclease recognition sequence
actgggnnnnn

SEQ ID NO: 33 - DNA, 10 bp, Artificial Sequence
  Description of Artificial SequenceBmrI type IIS palindromic restriction endonuclease recognition sequence complement
nnnncccagt

SEQ ID NO: 34 - DNA, 22 bp, Artificial Sequence
  Description of Artificial SequenceBpmI type IIS palindromic restriction endonuclease recognition sequence
ctggagnnnnnnnnnnnnnnnn

SEQ ID NO: 35 - DNA, 20 bp, Artificial Sequence
  Description of Artificial SequenceBpmI type IIS palindromic restriction endonuclease recognition sequence complement
nnnnnnnnnnnnnnctccag

SEQ ID NO: 36 - DNA, 22 bp, Artificial Sequence
  Description of Artificial SequenceBpuEI type IIS palindromic restriction endonuclease recognition sequence
cttgagnnnnnnnnnnnnnnnn

SEQ ID NO: 37 - DNA, 20 bp, Artificial Sequence
  Description of Artificial SequenceBpuEI type IIS palindromic restriction endonuclease recognition sequence complement
nnnnnnnnnnnnnncacaag

SEQ ID NO: 38 - DNA, 11 bp, Artificial Sequence
  Description of Artificial SequenceBsaI type IIS palindromic restriction endonuclease recognition sequence complement
nnnnngagacc

SEQ ID NO: 39 - DNA, 16 bp, Artificial Sequence
  Description of Artificial SequenceBseRI type IIS palindromic restriction endonuclease recognition sequence
gaggagnnnnnnnnnn

SEQ ID NO: 40 - DNA, 14 bp, Artificial Sequence
  Description of Artificial SequenceBseRI type IIS palindromic restriction endonuclease recognition sequence complement
nnnnnnnnctcctc

SEQ ID NO: 41 - DNA, 22 bp, Artificial Sequence
  Description of Artificial SequenceBsgI type IIS palindromic restriction endonuclease recognition sequence
gtgcagnnnnnnnnnnnnnnnn

SEQ ID NO: 42 - DNA, 20 bp, Artificial Sequence
  Description of Artificial SequenceBsgI type IIS palindromic restriction endonuclease recognition sequence complement
nnnnnnnnnnnnnngagacc

SEQ ID NO: 43 - DNA, 10 bp, Artificial Sequence
  Description of Artificial SequenceBspMI type IIS palindromic restriction endonuclease recognition sequence
acctgcnnnn

SEQ ID NO: 44 - DNA, 14 bp, Artificial Sequence
  Description of Artificial SequenceBspMI type IIS palindromic restriction endonuclease recognition sequence complement
nnnnnnnngcaggt

SEQ ID NO: 45 - DNA, 10 bp, Artificial Sequence
  Description of Artificial SequenceBssSI type IIS palindromic restriction endonuclease recognition sequence
cacgagnnnn

SEQ ID NO: 46 - DNA, 14 bp, Artificial Sequence
  Description of Artificial SequenceBssSI type IIS palindromic restriction endonuclease recognition sequence complement
nnnnnnnnctcgtg

SEQ ID NO: 47 - DNA, 16 bp, Artificial Sequence
  Description of Artificial SequenceBtgZI type IIS palindromic restriction endonuclease recognition sequence
gcgatgnnnnnnnnnn

SEQ ID NO: 48 - DNA, 20 bp, Artificial Sequence
  Description of Artificial SequenceBtgZI type IIS palindromic restriction endonuclease recognition sequence complement
nnnnnnnnnnnnnncatcgc

SEQ ID NO: 49 - DNA, 26 bp, Artificial Sequence
  Description of Artificial SequenceCstMI type IIS palindromic restriction endonuclease recognition sequence
aaggagnnnnnnnnnnnnnnnnnnnn

SEQ ID NO: 50 - DNA, 24 bp, Artificial Sequence
  Description of Artificial SequenceCstMI type IIS palindromic restriction endonuclease recognition sequence complement
nnnnnnnnnnnnnnnnnnctcctt

SEQ ID NO: 51 - DNA, 10 bp, Artificial Sequence
  Description of Artificial SequenceEarI type IIS palindromic restriction endonuclease recognition sequence complement
nnnngaagag

SEQ ID NO: 52 - DNA, 17 bp, Artificial Sequence
  Description of Artificial SequenceEciI type IIS palindromic restriction endonuclease recognition sequence
ggcggannnnnnnnnnn

SEQ ID NO: 53 - DNA, 15 bp, Artificial Sequence
  Description of Artificial SequenceEciI type IIS palindromic restriction endonuclease recognition sequence complement
nnnnnnnnntccgcc

SEQ ID NO: 54 - DNA, 31 bp, Artificial Sequence
  Description of Artificial SequenceEcoP15I type IIS palindromic restriction endonuclease recognition sequence
cagcagnnnnnnnnnnnnnnnnnnnnnnnnn

SEQ ID NO: 55 - DNA, 33 bp, Artificial Sequence
  Description of Artificial SequenceEcoP15I type IIS palindromic restriction endonuclease recognition sequence complement
nnnnnnnnnnnnnnnnnnnnnnnnnnnctgctg

SEQ ID NO: 56 - DNA, 18 bp, Artificial Sequence
  Description of Artificial SequenceRleAI type IIS palindromic restriction endonuclease recognition sequence
cccacannnnnnnnnnnn

SEQ ID NO: 57 - DNA, 15 bp, Artificial Sequence
  Description of Artificial SequenceRleAI type IIS palindromic restriction endonuclease recognition sequence complement
nnnnnnnnntgtggg

SEQ ID NO: 58 - DNA, 11 bp, Artificial Sequence
  Description of Artificial SequenceSapI type IIS palindromic restriction endonuclease recognition sequence complement
nnnngaagagc

SEQ ID NO: 59 - DNA, 13 bp, Artificial Sequence
  Description of Artificial SequenceUbaF2I type IIS palindromic restriction endonuclease recognition sequence
gaaaynnnnnrtg

SEQ ID NO: 60 - DNA, 13 bp, Artificial Sequence
  Description of Artificial SequenceUbaF2I type IIS palindromic restriction endonuclease recognition sequence complement
carnnnnnytttc

SEQ ID NO: 61 - DNA, 38 bp, Artificial Sequence
  Description of Artificial Sequencesynthetic exponential strand displacement amplification (SDA) of Factor V Leiden (FV) forward amplification primer (AP) oligonucleotide, FV forward AP
catcatgagagacatcgcctcctcagcaataggactac

SEQ ID NO: 62 - DNA, 38 bp, Artificial Sequence
  Description of Artificial Sequencesynthetic exponential strand displacement amplification (SDA) of Factor V Leiden (FV) reverse amplification primer (AP) oligonucleotide, FV reverse AP
aaattctcagaatttctgaacctcagcttcaaggacaa

SEQ ID NO: 63 - DNA, 20 bp, Artificial Sequence
  Description of Artificial Sequencesynthetic exponential strand displacement amplification (SDA) of Factor V Leiden (FV) reverse bumper primer oligonucleotide, FV reverse bumper
gccccattatttagccagga

SEQ ID NO: 64 - DNA, 21 bp, Artificial Sequence
  Description of Artificial Sequencesynthetic exponential strand displacement amplification (SDA) of Factor V Leiden (FV) biotinylated nest primer oligonucleotide, biotin-primer, FV nest primer
tgtaagagcagatccctggac

SEQ ID NO: 65 - DNA, 32 bp, Artificial Sequence
  Description of Artificial Sequencesynthetic NanoChip electronic microarray detection Factor V Leiden (FV) wild type single nucleotide polymorphism (SNP) discriminator oligonucleotide, FV Wt disc
ctgagtccgaacattgagtcctgtattcctcg

SEQ ID NO: 66 - DNA, 32 bp, Artificial Sequence
  Description of Artificial Sequencesynthetic NanoChip electronic microarray detection Factor V Leiden (FV) mutant single nucleotide polymorphism (SNP) discriminator oligonucleotide, FV Mut disc
gcagtatatcgcttgacatcctgtattccttg

SEQ ID NO: 67 - DNA, 23 bp, Artificial Sequence
  Description of Artificial Sequencesynthetic NanoChip electronic microarray detection Factor V Leiden (FV) stabilizer oligonucleotide, FV stab
cctgtccagggatctgctcttac

SEQ ID NO: 68 - DNA, 18 bp, Artificial Sequence
  Description of Artificial Sequencesynthetic NanoChip electronic microarray detection Factor V Leiden (FV) wild type single nucleotide polymorphism (SNP) fluorescence labeled reporter probe oligonucleotide, WT univ rep probe
ctcaatgttcggactcag

SEQ ID NO: 69 - DNA, 18 bp, Artificial Sequence
  Description of Artificial Sequencesynthetic NanoChip electronic microarray detection Factor V Leiden (FV) mutant single nucleotide polymorphism (SNP) fluorescence labeled reporter probe oligonucleotide, MUT univ rep probe
tgtcaagcgatatactgc

SEQ ID NO: 70 - DNA, 13 bp, Artificial Sequence
  Description of Artificial Sequencesynthetic real-time exponential strand displacement amplification (SDA) of Factor V Leiden (FV) mutant single nucleotide polymorphism (SNP) probe
cnnanggaacgga

SEQ ID NO: 71 - DNA, 13 bp, Artificial Sequence
  Description of Artificial Sequencesynthetic exponential strand displacement amplification (SDA) of Factor V Leiden (FV) wild-type single nucleotide polymorphism (SNP) probe
cnnanggagcgga

(19) United States
(12) Patent Application Publication
(10) Pub. No.: US 2009/0092967 A1
(43) Pub. Date: Apr. 9, 2009

(54) METHOD FOR GENERATING TARGET NUCLEIC ACID SEQUENCES

(75) Inventors: Zuxu Yao, San Diego, CA (US); Graham Lidgard, La Jolla, CA (US); Yevgeniy Belousov, Mill Creek, WA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Epoch Biosciences, Inc., Bothell, WA (US)

(21) Appl. No.: 11/768,418

(22) Filed: Jun. 26, 2007

Related U.S. Application Data
(60) Provisional application No. 60/805,847, filed on Jun. 26, 2006.

(51) Int. Cl. C12Q 1/68; C12P 19/34

(52) U.S. Cl. 435/6; 435/91.2

(57) ABSTRACT

The present invention provides methods of generating target nucleic acids for amplification using nicking enzymes and methods for amplifying the generated target nucleic acids.

### CROSS-REFERENCES TO RELATED APPLICATIONS

This application claims the benefit of U.S. Provisional Application No. 60/805,847, filed Jun. 26, 2006, the disclosure of which is hereby incorporated by reference in its entirety for all purposes.

### STATEMENT AS TO RIGHTS TO INVENTIONS MADE UNDER FEDERALLY SPONSORED RESEARCH OR DEVELOPMENT

Not Applicable

### REFERENCE TO A “SEQUENCE LISTING,” A TABLE, OR A COMPUTER PROGRAM LISTING APPENDIX SUBMITTED ON A COMPACT DISK

Not Applicable

### BACKGROUND OF THE INVENTION

Exponential strand displacement amplification (SDA) was disclosed in U.S. Pat. No. 5,455,166 requiring an initial denaturation of the target into single-stranded DNA (ssDNA), generation of hemiphosphorothioate sites which allow single strand nicking by restriction enzymes and extension by a polymerase lacking 5′-3′ exonuclease activity. U.S. Pat. No. 5,624,825 disclosed the simultaneous detection of more than one target and the requirement of at least one modified deoxynucleoside triphosphate (dNTP).

U.S. Pat. No. 5,648,211 describes the use of thermostable enzymes in SDA requiring either the generation of a hemimodified restriction site during amplification or the use of a substituted deoxynucleoside triphosphate. α-Boronated deoxynucleoside triphosphates, when incorporated into a double-stranded DNA (dsDNA), generates a restriction endonuclease recognition/cleavage site allowing a single nick in one DNA strand (U.S. Pat. No. 5,702,926). An isothermal in situ strand displacement amplification utilizing exposure to dry heat, restriction endonuclease pretreatment and mild depurination is disclosed byNuovo (Diagnostic Molec. Pathol., 9: 195-202 (2000)).

An abasic site endonuclease amplification assay was disclosed in U.S. Patent Application No. 2004/0101893. The use of this assay as a post amplification detection system in combination with other amplification systems, were also disclosed. All these assays require a denaturation step of dsDNA.

A few nucleases cut just one strand of DNA thereby introducing a nick into DNA (Besnier and Kong, EMBO Reports, 21: 782-786 (2001)). Most such proteins are involved in DNA repair and other DNA-related metabolism and cannot easily be used to manipulate DNA. They usually recognize long sequences and associate with other proteins to form active complexes that are difficult to manufacture (Higashitani et al., J. Mol. Biol., 237: 388-4000 (1994)). Single strand nicking endonucleases which nick only one strand of the DNA double strands and traditional restriction endonucleases are listed and updated in the REBASE Database (Roberts et al., Nucl. Acids Res., 31: 418-420 (2003)). Engineering of a nicking endonuclease has been described (Xu et al, PNAS USA 98: 12990-12995 (2001)). Isothermal assays using nicking enzymes, but still requiring a thermal denaturation step are described by Ehses et al., J. Biochem. Biophys. 63:170-186 (2005).

What is needed in the art is an isothermal assay which combines the advantages of target nucleic acid cycling, without the requirement for dsDNA denaturation and the use of modified dTNPs, retaining binding stability of the probe, an exquisitely specific cleavage site, the possibility for essentially instantaneous and highly sensitive reporter detection and the ability to directly combine detection with amplification procedures. Accordingly, there remains a need for compositions and methods that enable efficient detection of target nucleic acids with exquisite specificity. The present invention fulfills this need and others.

### BRIEF SUMMARY OF THE INVENTION

The present invention provides methods and compositions for improved hybridization and mismatch discrimination by isothermal amplification. In the practice of the invention, dsDNA is amplified isothermally by single strand displacement in the presence of a polymerase and a nicking enzyme that cuts on strand allowing the isothermal strand displacement. The method involves 1) the isolation of the target nucleic acids from a sample, 2) providing a mixture comprising a) a nicking endonuclease enzyme, b) a nucleic acid polymerase, c) deoxynucleosidetriphosphates and at least one primer which is complementary to a region at the 3′-end of a target fragment and further wherein each primer has a recognition sequence for the nicking endonuclease upstream of the 5′-end, allowing a time sufficient to generate nicks in one strand, the initiation points for isothermal strand displacement and the generation of reaction products.

One embodiment of the invention provides a method for generating a target nucleic acid sequence for amplification. The method comprises (a) providing a double stranded target sequence; (b) nicking one strand of said target sequence with a nicking enzyme, thereby generating the target nucleic acid sequence without thermal denaturation of the double stranded target sequence. The recognition site of the nicking enzyme: (i) is at least 6 nucleotides in length, (ii) is present in one strand of the target sequence about 1 to about 50 times, or (iii) comprises a combination of (i) and (ii). In some embodiments, the recognition site of the nicking enzyme is at least 6, 7, 8, 9, 10, 11, 12, 13, 14, 15, or 16 nucleotides in length. In some embodiments, the recognition site of the nicking enzyme is present in one strand of the target sequence about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 times. In some embodiments, the nicking enzyme is a type IIS nicking enzyme (e.g., a nicking enzyme selected from: Nt.BbvCI, Nb.BsmI, N. BbvC IA, N.BbvC IB, N.BstNB I, N.Alw I, Nb.Bpu101, N.Bst9I, NMlyI, R.BbvCI, Nb.SapI-1 (variant 33) and Nb.SapI-1 (E250K)). In some embodiments, the nicking enzyme is a modified type IIS nicking enzyme.

Another embodiment of the invention provides a method for amplifying a target nucleic acid sequence. The method comprises (a) generating a target nucleic acid sequence according to the method of claim 1; (b) contacting a first extension primer and a first bumper primer with the target nucleic acid sequence under conditions sufficient to allow first extension primer to hybridize to the target nucleic acid sequence and for the first bumper primer to hybridize to the target nucleic sequence at a site 5′ to the binding site of the first extension primer, wherein the 3′ end of the first extension primer comprises a target binding sequence and the 5′ end of the first extension primer comprises: (i) a recognition sequence for the nicking enzyme and (ii) a sequence which is complementary to the target nucleic acid, (c) simultaneously extending the first extension primer and the first bumper primer with a polymerase to produce a first extension product and a first bumper extension product that displaces the first extension product; (d) contacting a second extension primer and a second bumper primer with the displaced first extension product under conditions sufficient to allow the second extension primer to hybridize to the first extension product and for the second bumper primer to hybridize to the first extension product at a site 5′ to the binding site of the second extension primer, wherein the 3′ end of the second extension primer comprises a sequence that binds to the first extension product and the 5′ end of the second extension primer comprises: (i) a recognition sequence for the nicking enzyme and (ii) a sequence which is complementary to the target nucleic acid; and (e) simultaneously extending the second extension primer and the second bumper primer with the polymerase to produce a second extension product and a second bumper extension product that displaces the second extension product, thereby generating an amplified target sequence. In some embodiments, the polymerase is a DNA polymerase without 5′→3′ exonuclease activity (e.g., a polymerase selected from: Bst DNA Polymerase Large Fragment, Bca DNA polymerase, Klenow fragment of DNA polymerase I, Phi29 DNA polymerases, Sequenase 2.0 T7 DNA Polymerase and T5 DNA polymerase). In some embodiments, the method further comprises (f) contacting the first extension primer to the second extension product under conditions sufficient to allow the first extension primer to hybridize to the second extension product and extending the first extension primer with the polymerase to generate a double stranded product comprising restriction sites recognized by the nicking enzyme; (g) contacting the double stranded product with the nicking enzyme under conditions sufficient to allow the nicking enzyme to cleave a single strand of the double stranded product, thereby generating a nicked double stranded product with a nick site on each strand; (h) contacting the first and second extension primer with the nicked double stranded product under conditions sufficient to allow the first and second extension primers to hybridize to the nicked double stranded product; and (i) extending the first and second extension primers with a polymerase, thereby releasing single stranded amplified target sequences into solution. In some embodiments, the method further comprises (h) detecting the amplified target sequence. In some embodiments, the amplified target sequence is detected by: (i) contacting the amplified target sequence with AP site probe and an AP endonuclease under conditions sufficient to allow the AP site probe to hybridize to the amplified target nucleic acid, wherein the AP site probe comprises an oligonucleotide NA that hybridizes to the amplified target nucleic acid and a functional tail R comprising a detectable reporter group, the functional tail R attached via a phosphodiester bond of a phosphate group to the 3′ terminal nucleotide of the NA, wherein the reporter group is not detected when the functional tail R is attached to the NA; and (j) incubating the amplified target nucleic acid sequence, the AP site probe, and the AP endonuclease under conditions sufficient to allow the AP endonuclease to cleave the phosphodiester bond attaching the functional tail R to the 3′ terminal nucleotide of the NA, wherein the AP endonuclease preferentially cleaves the phosphodiester bond attaching the tail R to the NA when the NA is hybridized with a complementary target nucleic acid sequence in comparison to when the NA is unhybridized or hybridized to a non-complementary nucleic acid; and (k) detecting the reporter group on the cleaved functional tail R, whereby the amplified target nucleic acid sequence is detected. In some embodiments, the method further comprises contacting the amplified target nucleic acid sequence with an enhancer oligonucleotide, wherein the 5′-end of the enhancer oligonucleotide hybridizes to the amplified target nucleic acid sequence about 0 to about 5 bases 3′ to the site where the AP site probe hybridizes to the amplified target nucleic acid sequence. In some embodiments, the 5′-end of the AP site probe is covalently linked to the 3′-end of the enhancer. In some embodiments, a quencher molecule is attached to the 5′ end of the NA of the AP site probe via a non-cleavable linker. In some embodiments, cleavage of the phosphodiester bond results in a hybridized NA having a free 3′-OH. In some embodiments, the amplified target nucleic acid sequence, the AP site probe, and the AP endonuclease are incubated under conditions sufficient to simultaneously allow the AP endonuclease to cleave the phosphodiester bond of the AP site probe and the polymerase to extend the cleaved AP site probe in a template-specific manner. In some embodiments, the NA of the AP site probe is 3-200, 5-150, 7-100, 10-50, 15-40, or 20-30 nucleotides in length. In some embodiments, the AP site probe further comprises at least one modified base. In some embodiments, the functional tail R is attached to the phosphate group through a hydroxyprolinol linker. In some embodiments, the reporter group is a fluorophore. In some embodiments, the AP endonuclease is a Class II AP endonuclease (e.g., an E. coli Endonuclease IV).

A further embodiment of the invention provides kits comprising at least one of the nicking enzymes, primers, probes, and DNA polymerases described herein.

### BRIEF DESCRIPTION OF THE DRAWINGS

FIG. 1 is a diagram illustrating the method of the invention to perform true isothermal amplification of dsDNA. FIG. 1A illustrates the isothermal generation of ssDNA template. FIG. 1B illustrates the initial steps of SDA and FIG. 1C illustrates the exponential cycles in SDA.

FIG. 2 shows an example of an endonuclease IV signal detection system.

FIG. 3 shows real-time homogeneous amplification (SDA) and detection of Mycobacterium tuberculosis DNA using an endonuclease IV probe. Fluorescence is plotted versus time for various amounts of input target.

FIG. 4 shows post-amplification genotype analysis of isothermal 1-step SDA product on NanoChip® microarray.

FIG. 5 shows Real time genotype analysis of isothermal 1-step SDA products from human genomic DNA using MGB Eclipse® genotyping probes (Nanogen, Bothell, Wash.) FIG. 5A represents the analysis of homozygous wild type samples. FIG. 5B represents analysis of mutant samples, respectively, FIG. 5C represents the analysis of a heterozygous sample. FIG. 5D represents the no template control.

### DETAILED DESCRIPTION OF THE INVENTION

### I. General

The present invention provides assay methods that combine the advantages of nucleic acid amplification cycling with a nicking enzyme that cuts one strand of dsDNA allowing the isothermal strand displacement generation of single strand DNA eliminating the need for thermal denaturation, and providing methods with efficient, flexible and simpler amplification protocols.

### II. Definitions

As use in herein a “nick” is a point in a double stranded DNA molecule where there is no phosphodiester bond between adjacent nucleotides of one strand. A nick is typically induced by damage or caused by enzyme action.

A “nicking endonuclease” or “nicking enzyme” is an enzyme that specifically introduces a nick in one of the strands in a double-stranded nucleic acid (e.g., DNA).

A “restriction endonuclease” is a class of bacterial enzymes that cut both strands of DNA at specific sites.

An “amplicon sequence” is the target sequence that is exponentially amplified and contains primer specific sequences.

“Isothermal SDA amplification” or “isothermal strand displacement amplification” as used in here refers to amplification where no thermal denaturation of double stranded DNA (dsDNA) (e.g., at 95° C. or other elevated temperature) is used to generate single stranded DNA (ssDNA). The isothermal SDA amplification of the present refers the generation of ssDNA by a polymerase with strand displacement ability starting from a nicking site, allowing amplification by the polymerase.

An “extension primer” is a primer that is extended during an SDA reaction.

A “bumper primer” is a primer used to displace primer extension products in SDA reaction. The bumper primer anneals to a target sequence upstream of the extension primer such that extension of the bumper primer displaces the downstream extension primer and its extension product.

An abasic site is an naturally occurring Apurinic/Apyrimidinic (AP) site in a nucleic acid sequence or a synthetic linker that is recognized and cleaved by Class II AP endonucleases when it appears in double stranded DNAs.

As used herein, an AP endonuclease refers to an enzyme that binds to and cleaves the phosphodiester backbone at an abasic (AP) site on a nucleic acid strand in a double stranded DNA. Preferred AP endonucleases cleave the phosphodiester backbone on the 5′ side of the AP site via a hydrolytic mechanism that provides a free 3′-OH group that serves as a substrate for DNA polymerases.

### III. Isothermal Strand Displacement Amplification

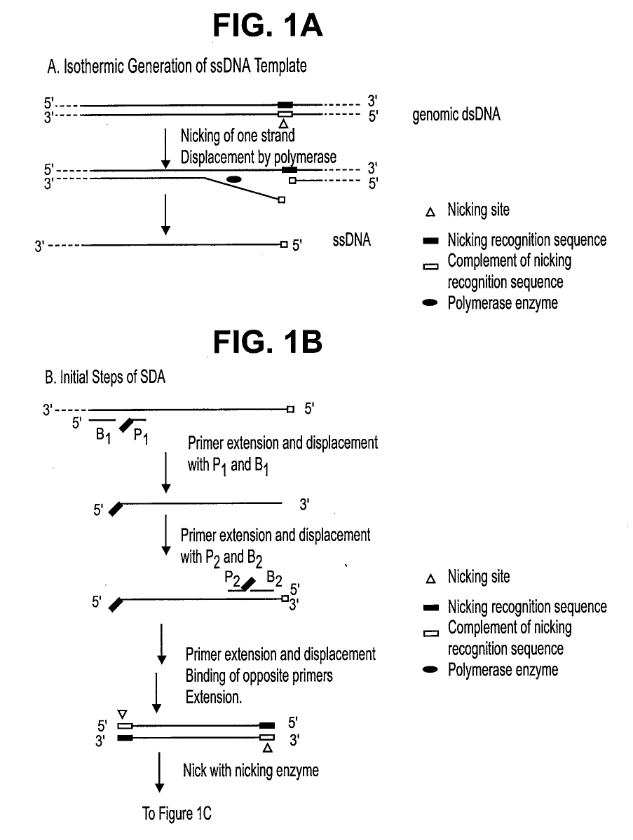

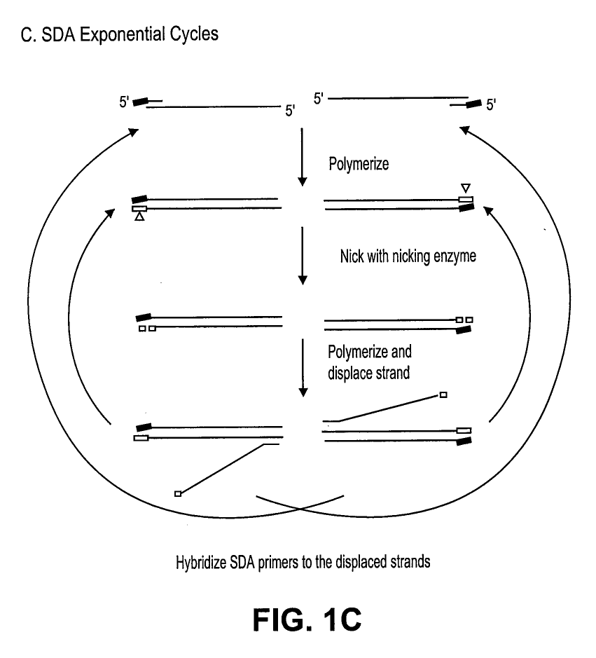

SDA amplification after generation of a ssDNA by thermal denaturation has been disclosed (U.S. Pat. Nos. 5,455,166; 5,624,825; 5,624,825 and Walker et al., Nucl. Acids Res., 24: 348-353 (1996)) as illustrated in FIG. 1b) and c). The generation of ssDNA after nicking by a nicking endonuclease and displacement by DNA polymerase lacking 5′ to 3′ exonuclease activity is illustrated in FIG. 1a).

In contrast to traditional “isothermal SDA” which require at least one thermal denaturation step at about 95° C. to generate single stranded DNA (ssDNA), the isothermal SDA of the invention generates ssDNA isothermally by polymerase strand displacement and, accordingly eliminates the thermal denaturation step. The isothermal generation of single stranded DNA, requires the involvement of a nicking enzyme that catalyzes a nick in one of the DNA strands, the initiation point for strand displacement by the polymerase.

According to the methods of the invention, a single stranded target nucleic acid sequence is generated by contacting a double-stranded target nucleic acid sequence (e.g., dsDNA) with a nicking enzyme (e.g., a type IIs modified nicking enzyme as described herein).

Three issues are of importance to facilitate the production of ssDNA for SDA: i) the frequency of the nicking enzyme recognition sequence in the target gene sequence, ii) the length of the nicking recognition sequence and iii) the orientation of the nicking enzyme's recognition sequence. The frequency of the nicking enzyme recognition sequences in the target is such that the strand displaced generated ssDNA sequences are equal or greater than that of the amplicon sequence. The nicking enzyme recognition site is at least 6, 7, 8, or 9 nucleotides in length. In some embodiments, the nicking enzyme recognition site occurs in the target sequence about 1 to about 50, about 2 to about 40, about 3 to about 35, about 4 to about 30, about 5 to about 25, about 6 to about 20, about 10 to about 15 times. In some embodiments, the nicking enzyme recognition site is located about 1 to about 10,000, about 2 to about 5,000, about 3 to about 2,500, about 4 to about 1250, about 5 to about 1,000, about 10 to about 500, about 15 to about 250, about 20 to about 150 nucleotides from the binding site for a probe to detect the amplified target nucleic acid. In some embodiments, the nicking enzyme is selected from the enzymes disclosed in Table 1.

In some embodiments, the recognition site sequence of the nicking enzyme is located upstream of the 5′-end of the targeted sequence. Strand displacement from the nicking site, by the polymerase lacking 5′ to 3′ exonuclease activity with good strand displacement characteristics, will therefore produce single strand targets that will include the probe recognition sequence.

In one embodiment the isothermal SDA of the present invention requires at least one nicking endonuclease. A preferred embodiment utilizes a first and a second nicking enzymes, one set of primers containing a recognition sequence located upstream of the 5′-end of the probe sequence for one of said nicking enzymes.

In one embodiment target nucleic acid by strand displacement amplification is performed by using a thermophilic nicking enzyme, a thermophilic DNA polymerase and a thermophilic endonuclease IV. In another embodiment at least one of the three enzymes is thermophilic. The use of a thermophilic DNA polymerase and a thermophilic restriction endonuclease was previously disclosed in U.S. Pat. No. 5,648,211.

In another embodiment SDA is performed in the presence of a nicking enzyme, a restriction enzyme, generated hemimodified restriction site and a polymerase lacking 5′-3′ exonuclease activity. A hemimodified restriction site is generated during amplification utilizing at least one dNTP selected from dNTPαS (U.S. Pat. No. 5,455,166), a modified dNTP (U.S. Pat. No. 5,624,825) or α-boronated deosynucleoside triphosphate (U.S. Pat. No. 5,648,211). In the above embodiment, the nicking enzyme generates nicks for the initiation of strand displacement, allowing isothermal generation of single stranded DNA target. Amplification primers containing a restriction recognition sequence upstream of the 5′-end of the primer recognition sequence, allow the generation of hemimodified restriction modified site using an appropriate modified dTNP.

A. Nicking Enzymes

Nicking enzymes can be isolated or genetically engineered from restriction enzymes (Xu et al, PNAS 98: 12990-12995 (2001)). A number of nicking enzymes are available either commercially or have been disclosed. The following Table contains a list of nicking enzymes useful to perform the isothermal amplification of the current inventions.

TABLE I

Nicking enzymes and their specificity.

| | | |
|---|---|---|
| Enzyme | Nicking Specificity | Source |

| | | |
|---|---|---|
| Nb.BsmI | 5′ . . . GAATGCN . . . 3′ | http://www.neb.com |
| | 3′ . . . CTTAC▴GN . . . 5′ | |
| N. BbvC IA | 5′ . . . GC▾TGAGG . . . 3′ | http://www.neb.com |
| | 3′ . . . CGACTCC . . . 5′ | |
| N.BbvC IB | 5′ . . . CC▾TCAGC . . . 3′ | http://www.neb.com |
| | 3′ . . . GGAGTCG . . . 5′ | |
| N.BstNB I | 5′ . . . GAGTCNNNN▾N . . . 3′ | http://www.neb.com |
| | 3′ . . . CTCAGNNNNN . . . 5′ | |
| N.Alw I | 5′ . . . GGATCNNNN▾N . . . 3′ | http://www.neb.com |
| | 3′ . . . CCTAGNNNNN . . . 5′ | |
| Nb.Bpu101 | 5′ . . . GC▾TNAGG . . . 3′ | http://www.fermentas.com |
| | 3′ . . . CGANTCC . . . 5′ | U.S. Pat. No. 6,867,028 |
| N.Bst9I | 5′ . . . GAGTCNNNN▾N . . . 3′ | http://www.sibenzyme.com |
| | 3′ . . . CACAGNNNNN . . . 5′ | |
| NMlyI | 5′ . . . GAGTCNNNNN▾N . . . 3 | Besnier &Kong, EMBO Reports, 21: |
| | 3′ . . . CTCAGNNNNNN . . . 5′ | 782-786 (2001). |
| R.BbvCI | 5′ . . . CCTCAGC . . . 3′ | Heiter et al., JMB., 348: 631-640 |
| | 3′ . . . GG▴AGTCG . . . 5′ | (2005) |
| | 5′ . . . CC▾TCAGC . . . 3′ | |
| | 3′ . . . GGAGTCG . . . 5′ | |
| Nb.SapI-1 | 5′ . . . GCTCTTCNNNNN . . . 3′ | Samuelson et al., NAR., 32: 3661- |
| (variant 33) | 3′ . . . CGAGAAGNNNN▴N . . . 5′ | 3671 (2004) |
| Nb.SapI-1 | 5′ . . . GCTCTTCN▾N . . . 3′ | |
| (E250K) | 3′ . . . CGAGAAGNN . . . 5′ | |

▴ or ▾ indicates the nicking position in the sequence.

There are more than eighty type of IIA/IS restriction endonuclease with different known recognition specificities. A novel genetic screening method was devised to convert type IIS restriction endonucleases into strand-specific nicking endonucleases (Zhu et al, J Mol. Biol., 337: 573-83 (2005)). A selected preferred list of these restriction enzymes and their recognition sequences are listed in Table II. These type IIS restriction enzymes can potentially be converted by the Zhu et al protocol to nicking enzymes.

TABLE 2

Palindromic Restriction Endonucleases.

| | | | |
|---|---|---|---|
| Enzymes | Recognition Sequence | Enzymes | Recognition Sequence |

| | | | |
|---|---|---|---|
| Aar I | 5′ . . . CACCTGC(N)₄▾ . . . 3′ | BseY I | 5′ . . . C▾CCAGC . . . 3′ |
| | 3′ . . . GTGGACG(N)₈▴ . . . 5′ | | 3′ . . . GGGTC▴G . . . 5′ |
| Ace III | 5′ . . . CAGCTC(N)₇▾ . . . 3′ | Bsg I | 5′ . . . GTGCAG(N)₁₆▾ . . . 3′ |
| | 3′ . . . GTCGAG(N)₁₁▴ . . . 5′ | | 3′ . . . CCAGAG(N)₁₄▴ . . . 5′ |
| Acu I | 5′ . . . CTGAAG(N)₁₆▾ . . . 3′ | Bsm I | 5′ . . . GAATGCN▾ . . . 3′ |
| | 3′ . . . GACTTC(N)₁₄▴ . . . 5′ | | 3′ . . . CCAGA▴GN . . . 5′ |
| Alw I | 5′ . . . GGATC(N)₄▾ . . . 3′ | BspM I | 5′ . . . ACCTGC(N)₄▾ . . . 3′ |
| | 3′ . . . CCTAG(N)₅▴ . . . 5′ | | 3′ . . . TGGACG(N)₈▴ . . . 5′ |
| Bbr7 I | 5′ . . . GAAGAC(N)₇▾ . . . 3′ | BsrB I | 5′ . . . CCG▾CTC . . . 3′ |
| | 3′ . . . CTTCTG(N)₁₁▴ . . . 5′ | | 3′ . . . GGC▴GAG . . . 5′ |
| Bbs I | 5′ . . . GAAGAC(N)₂▾ . . . 3′ | BsrD I | 5′ . . . GCAATG(N)₂▾ . . . 3′ |
| | 3′ . . . CTTCTG(N)₆▴ . . . 5′ | | 3′ . . . CGTTAC▴N . . . 5′ |
| Bbv I | 5′ . . . GCAGC(N)₈▾ . . . 3′ | BssS I | 5′ . . . C▾ACGAG(N)₄ . . . 3′ |
| | 3′ . . . CGTCG(N)₁₂▴ . . . 5′ | | 3′ . . . GTGCT▴C(N)₈ . . . 5′ |
| BbvC I | 5′ . . . CC▾TCAGC . . . 3′ | BtgZ I | 5′ . . . GCGATG(N)₁₀▾ . . . 3′ |
| | 3′ . . . GGAGT▴CG . . . 5′ | | 3′ . . . CGCTAC(N)₁₄▴ . . . 5′ |
| Bcc I | 5′ . . . CCATC(N)₄▾ . . . 3′ | Bts I | 5′ . . . GCAGTG(N)₂▾ . . . 3′ |
| | 3′ . . . GGTAG(N)5▴ . . . 5′ | | 3′ . . . CGTCAC▴N . . . 5′ |
| BciV I | 5′ . . . GTATCC(N)₆▾ . . . 3′ | CstM I | 5′ . . . AAGGAG(N)₂₀▾ . . . 3′ |
| | 3′ . . . CATAGG(N)₅▴ . . . 5′ | | 3′ . . . TTCCTC(N)₁₈▴ . . . 5′ |
| BfuA I | 5′ . . . ACCTGC(N)₄▾ . . . 3′ | Drd II | 5′ . . . GAACCA▾ . . . 3′ |
| | 3′ . . . TGGACG(N)₈▴ . . . 5′ | | 3′ . . . CTTGGT▴ . . . 5′ |
| BmgB I | 5′ . . . CAC▾GTC . . . 3′ | Ear I | 5′ . . . CTCTTC(N)₁▾ . . . 3′ |
| | 3′ . . . GTG▴CAG . . . 5′ | | 3′ . . . GAGAAG(N)₄▴ . . . 5′ |
| Bmr I | 5′ . . . ACTGGG(N)₅▾ . . . 3′ | Eci I | 5′ . . . GGCGGA(N)₁₁▾ . . . 3′ |
| | 3′ . . . TGACCC(N)₄▴ . . . 5′ | | 3′ . . . CCGCCT(N)₉▴ . . . 5′ |
| Bpm I | 5′ . . . CTGGAG(N)₁₆▾ . . . 3′ | EcoP15 I | 5′ . . . CAGCAG(N)₂₅▾ . . . 3′ |
| | 3′ . . . GACCTC(N)₁₄▴ . . . 5′ | | 3′ . . . GTCGTC(N)₂₇▴ . . . 5′ |
| Bpu10 I | 5′ . . . CC▾TNAGC . . . 3 | Pfl1108 I | 5′ . . . TCGTAG▾ . . . 3′ |
| | 3′ . . . GGANT▴CG . . . 5′ | | 3′ . . . AGCATC₇▴ . . . 5′ |
| BpuE I | 5′ . . . CTTGAG(N)₁₆▾ . . . 3′ | RleA I | 5′ . . . CCCACA(N)₁₂▾ . . . 3′ |
| | 3′ . . . GAACAC(N)₁₄▴ . . . 5′ | | 3′ . . . GGGTGT(N)₉▴ . . . 5′ |
| Bsa I | 5′ . . . GGTCTC(N)₁▾ . . . 3′ | Sap I | 5′ . . . GCTCTTC(N)₁▾ . . . 3′ |
| | 3′ . . . CCAGAG(N)₅▴ . . . 5′ | | 3′ . . . CGAGAAG(N)₄▴ . . . 5′ |
| Bsb I | 5′ . . . CAACAC . . . 3′ | UbaF2 I | 5′ . . . GAAAY(N)₅RTG▾ . . . 3′ |
| | 3′ . . . GTTGTG . . . 5′ | | 3′ . . . CTTTY‘N)₅R‘AC▴ . . . 5′ |
| | | | R = A or G; Y = C or T |
| BseR I | 5′ . . . GAGGAG(N)₁₀▾ . . . 3′ | UbaP I | 5′ . . . CGAACG▾ . . . 3′ |
| | 3′ . . . CTCCTC(N)₈▴ . . . 5′ | | 3′ . . . GCTTGC₇▴ . . . 5′ |

B. DNA Polymerase

DNA polymerases useful in this method include those that are capable of extending from the nick while displacing the down stream strand. Importantly, the polymerase should lack any 5′ to 3′ exonuclease activity. Useful DNA polymerases are Bst DNA Polymerase Large Fragment (New England Biolabs, Ipswich, Mass.), Bca DNA polymerase (Takara Shuzo, Shiga., Japan), Klenow fragment of DNA polymerase I, for example Klenow Fragment, exo-, Phi29 DNA polymerases (FERMENTAS, Hanover, Md.), Sequenase 2.0 T7 DNA Polymerase (Amersham Biosciences Corp, Piscataway, N.J.), T5 DNA polymerase. Preferred DNA polymerases are Bst and Bca DNA polymerases.

C. Additional Assay Components

The reaction mixture also typically comprises appropriate buffers (e.g., phosphate buffers), a source of magnesium ions (e.g., MgCl₂), and dNTPs (dATP, dGTP, dCTP and TTP) 50 nM forward primer, 500 nM reverse primer, 50 nM each bumper, 4U Bst DNA polymerase, 4U BbvC1B (New England Biolabs), 0.1U Endo IV (Trevigen) diluted in Diluent A (New England Biolabs). After addition of all above components, including the three enzymes at room temperature, reaction tubes were placed directly in the thermocycler and were incubated at 49° C. for 50 min.

D. Detection Systems

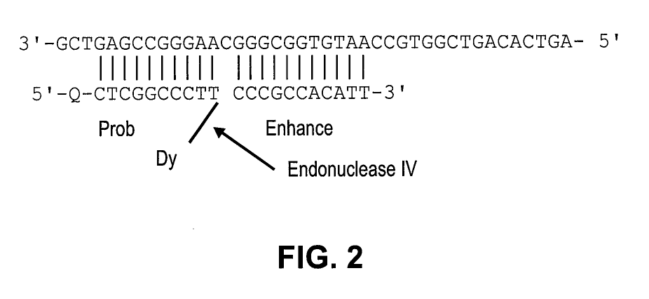

The target amplified by the isothermal strand displacement method of the current invention can be detected in numerous ways. In one preferred embodiment, the isothermal strand displacement method is coupled to the endonuclease IV signal amplification method. The endonuclease IV signal amplification method is disclosed in U.S. Patent Publication No. 2003/026133, which is hereby incorporated herein by reference in its entirety. This method requires i) a short FRET probe, with a quencher preferably at the 5′-end and fluorescent dye coupled to 3′-hydroxyl through a phosphate bond and rigid linker; ii) a shorter enhancer oligonucleotide and iii) an endonuclease IV enzyme. Both the probe and the enhancer are complementary to the target or amplified target, hybridizing in such a fashion that there is a one base gap between probe and enhancer, generating a substrate for the endonuclease IV, which specifically hydrolyzes the phosphate linkage between the oligonucleotice and the fluorescent dyed (FIG. 2). The endonuclease IV does not hydrolyze unhybridized FRET probes. In some embodiments the SDA amplified target is detected with the FRET probe and the endonuclease IV without the enhancer.

In other embodiments the amplified isothermal target of the invention is detected by a probe that fluoresces on hybridization to its complementary target sequence. Minor groove binder-based probes that fluoresce on hybridization have been disclosed in U.S. Patent Publications Nos. 2003/0175728 and 2005/0214797 which is hereby incorporated herein by reference in its entirety. Molecular beacon probes, containing hairpin-stem regions have been disclosed in U.S. Pat. No. 5,312,728. A cleavable hairpin probe was disclosed (Nadeau et al., Anal. Biochem., 276: 177-187 (1999)) to detect SDA amplification product. In some embodiments the isothermally amplified target of the inventions is detected directly or indirectly by labeled or FRET labeled primers.

In some embodiments, the amplified target of isothermal SDA of this invention is detected by primer-based methods. Primer-based detection methods are either indirect or direct. Indirect detection is performed by using biotinylated primers in a sandwich format utilizing a biotin-straptavidin capture and a detection probe or a variation thereof. Direct primer-based detection typically require a hairpin FRET primer that when hybridized to its complementary extend target strand, fluoresces (U.S. Pat. No. 6,656,680).

In some embodiments, the amplified target of isothermal SDA of this invention is detected using an AP site probe as described in. e.g., U.S. Patent Publication No. 2004/0101893.

Generally, the structure of an AP site probe is as follows:

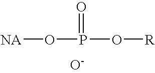

An AP site probe is comprised of a nucleic acid (“NA”) covalently bound by its 3′-terminal oxygen atom to a functional, chemical tail (“R”) through a phosphodiester group.

The number of nucleotides in the NA component can be 3 to 200, 3 to 100 or 3 to 200 nucleotides in length, depending on the intended use. Usually, the length of the NA is from 5 to 30 nucleotides. More typically, the length of the NA is 6-25, 7-20, or 8-17 nucleic acids. Most often, the NA component is about 6, 7, 8, 9, 10, 11, 12, 13, 14, 15 or 16 nucleic acids in length. Usually, the NA component will have a hybridization melting temperature of about 10 to 80° C., more typically of about 20 to 70° C., and preferably about 30° C., 40° C., 50° C. or 60° C.

The sugar, or glycoside, portion of the NA component of the conjugates can comprise deoxyribose, ribose, 2-fluororibose, and/or 2-O-alkyl or alkenylribose wherein the alkyl group comprises 1 to 6 carbon atoms and the alkenyl group comprises 2 to 6 carbon atoms. In the naturally-occurring nucleotides, modified nucleotides and nucleotide analogues that can comprise an oligonucleotide, the sugar moiety forms a furanose ring, the glycosidic linkage is of the beta configuration, the purine bases are attached to the sugar moiety via the purine 9-position, the pyrimidines via the pyrimidine 1-position and the pyrazolopyrimidines via the pyrazolopyrimidine 1-position (which is equivalent to the purine 9-position). In a preferred embodiment, the sugar moiety is 2-deoxyribose; however, any sugar moiety known to those of skill in the art that is compatible with the ability of the oligonucleotide portion of the compositions of the invention to hybridize to a target sequence can be used.

In one preferred embodiment, the NA is DNA. An AP site probe comprising DNA can be used to detect DNA, as well as RNA, targets. In another embodiment, the NA is RNA. An AP site probe comprising RNA is generally used for the detection of target DNAs. In another embodiment, an AP site probe can contain both DNA and RNA distributed within the probe. In mixed nucleic acid probes, DNA bases preferably are located at 3′-end of the probe while RNA bases are at the 5′-end. It is also preferred when the 3′-terminal nucleotide is 2′-deoxyribonucleotide (DNA) and when at least four 3′-terminal bases of NA are DNA bases.

Usually, the NA component contains the major heterocyclic bases naturally found in nucleic acids (uracil, cytosine, thymine, adenine and guanine). In some embodiments, the NA contains nucleotides with modified, synthetic or unnatural bases, incorporated individually or multiply, alone or in combination. Preferably, modified bases increase thermal stability of the probe-target duplex in comparison with probes comprised of only natural bases (i.e., increase the hybridization melting temperature of the probe duplexed with a target sequence). Modified bases include naturally-occurring and synthetic modifications and analogues of the major bases such as, for example, hypoxanthine, 2-aminoadenine, 2-thiouracil, 2-thiothymine, inosine, 5-N⁴-ethenocytosine, 4-aminopyrrazolo[3,4-d]pyrimidine and 6-amino-4-hydroxy-[3,4-d]pyrimidine. Any modified nucleotide or nucleotide analogue compatible with hybridization of an AP site probe with a target nucleic acid conjugate to a target sequence is useful in the practice of the invention, even if the modified nucleotide or nucleotide analogue itself does not participate in base-pairing, or has altered base-pairing properties compared to naturally-occurring nucleotides. Examples of modified bases are disclosed in U.S. Pat. Nos. 5,824,796; 6,127,121; 5,912,340; and PCT Publications WO 01/38584; WO 01/64958, each of which is hereby incorporated herein by reference in its entirety. Preferred modified bases include 5-hydroxybutynyl uridine for uridine; 4-(4,6-Diamino-1H-pyrazolo[3,4-d]pyrimidin-3-yl)-but-3-yn-1-ol, 4-amino-1H-pyrazolo[3,4-d]pyrimidine, and 4-amino-1H-pyrazolo[3,4-d]pyrimidine for adenine; 5-(4-Hydroxy-but-1-ynyl)-1H-pyrimidine-2,4-dione for thymine; and 6-amino-1H-pyrazolo[3,4-d]pyrimidin-4(5H)-one for guanine. Particularly preferred modified bases are “Super A™,” “Super G™: 4-hydroxy-6-amino pyrazolopyrimidine” (www.nanogen.com) and “Super T™”. Modified bases preferably support the geometry of a naturally occurring B-DNA duplex. Modified bases can be incorporated into any position or positions in an AP site probe, but preferably are not incorporated as the 3′-terminal base.

In another embodiment, some or all nucleotides of NA are substituted or contain independently different sugar-phosphate backbone modifications including 2′-O-alkyl RNA nucleotides, phosphorothioate internucleotide linkage, methylphosphonate, sulfamate (e.g., U.S. Pat. No. 5,470,967) and polyamide (i.e., peptide nucleic acids, PNA), LNA (locked nucleic acid), and the like. Such modifications and others of potential use in the present invention are described, for example, in Boutorine, et al., Biochimie 76:23 (1994); Agrawal, et al., Proc. Natl. Acad. Sci. 88:7595 (1991); Mag, et al., Nucleic Acids Res. 19:1437 (1991); Kurreck, Eur. J. Biochem. 270:1628 (2003); Lesnik, et al., Biochemistry 32:7832 (1993); Sproat, et al., Nucleic Acids Symp. Ser. 24:59 (1991); Iribarren, et al., Proc. Natl. Acad. Sci. 87:7747 (1990); Demidov, Trends Biotechnol. 21:4 (2003); Nielsen, Methods Mol. Biol. 208:3 (2002); Nielsen and Egholm, Curr. Issues Mol. Biol. 1:89 (1999); Micklefield, Curr. Med. Chem. 8:1157 (2001); Braasch, et al., Chem. Biol. 8:1 (2001); and Nielsen, Curr. Opin. Biotechnol. 12:16 (2001).

Within the scope of present invention, modifications of the bases and sugar-phosphate backbone as well as other functional moieties conjugated with the probe can serve to improve the sequence specificity of the target-probe duplex formation. In particular, binding between the probe and a matched target nucleic acid is detectably increased over binding to a mismatched target nucleic acid. By “matched target nucleic acid” is intended a target nucleic acid that contains a sequence that is completely complimentary to the probe sequence. By “mismatched target nucleic acid” is intended a polynucleotide that contains a sequence that is partially complimentary to the probe sequence such that it contains at least one mismatched, non-complimentary base, deletion or insertion in comparison to the probe sequence. For example, use of modified bases in an AP site probe allows for more stable base pairs than when using natural bases and enables the use of shorter probes for the same reaction conditions. Reduction of the probe length increases the ability of the probe to discriminate a target polymorphism as small as a Single Nucleotide Polymorphism (“SNP”) due to a proportional increase in the contribution of each duplex base pair to the overall duplex stability. In general, the shorter the probe, the greater the relative contribution of an individual base pair in to the overall duplex stability, and the better the probe discrimination of the target polynucleotide polymorphism.

The functional tail R enables detection of the endonuclease tail-cleavage reaction. The structure of R can be of any size and composition as long as it supports the template-specific, endonuclease tail-cleavage reaction. R can be as large as a natural protein with molecular mass up to 1,000,000 Daltons or it can be as small as a single atom (i.e., a radioactive isotope, such as a hydrogen or an iodine). Since the enzymatic hydrolysis occurs between the 3′-terminal oxygen atom of the NA and the phosphorus atom of the phosphodiester bond, for the purposes of the present invention, the phosphate moiety of the probe is considered a part of the functional tail R. For example, when R is hydrogen (R=—H), the functional tail of the probe is a phosphate moiety —P(O)(OH)₂ or —PO₃²⁻. The tail R can be hydrophobic or hydrophilic, electrically neutral, positively or negatively charged. It can be comprised of or include independently different functional groups, including mass tags, fluorescent or non-fluorescent dyes, linkers, radioisotopes, functional ligands like biotin, oligopeptides, carbohydrates and the like. For example, as demonstrated herein, Endonuclease IV from E. coli efficiently cleaves from the 3′-end of a probe bound to the target nucleic acid a relatively hydrophilic, negatively charged fluorescein moiety as well as an electrically neutral, hydrophobic quenching dye.

The tail R can contain components that improve specificity by blocking non-specific cleavage reactions in the absence of a target molecule without affecting the target-dependent, specific reaction. It is also within the scope of present invention that the tail R or some structural components of it can improve the specificity of the target-probe or enhancer-probe complementary binding so that the thermodynamic difference in the probe/enhancer binding to matched and mismatched target nucleic acids is increased. Examples of such structural components are minor groove binders (MBs).

The functional tail R can incorporate mono-, oligo- or polynucleotides. Nucleotide residues introduced into the tail structure are not intended to bind to the target nucleic acid.

In addition to a functional chemical tail R conjugated to the 3′-end of an AP site probe through a phosphodiester group, the probe optionally can contain other tails and functional moieties covalently attached to the probe or the tail via an appropriate linker. Preferably, the additional moieties do not interfere with endonuclease recognition of the AP tail-cleavage site or the template-specific tail-cleavage reaction. In one embodiment, additional moieties are attached to the 5′-end of the NA portion of the probe. In another embodiment, an additional moiety is conjugated to nucleotide bases of the probe such that, when the probe-target duplex is formed, the moieties are located within the major groove of the duplex.

Incorporation of a moiety in addition to the functional, chemical tail can serve to improve the probe hybridization properties. Examples of such moieties include minor groove binders and intercalators. Minor groove binders are described in U.S. Pat. Nos. 6,492,346 and 6,486,308, both of which are hereby incorporated herein by reference. In other embodiments, these moieties operate in conjunction with the functional tail R to aid in the detection of an endonuclease tail-cleavage reaction. Examples of such moieties include radioisotopes, radiolabelled molecules, fluorescent molecules or dyes, quenchers (dyes that quench fluorescence of other fluorescent dyes), fluorescent antibodies, enzymes, or chemiluminescent catalysts. Another suitable moiety is a ligand capable of binding to specific proteins which have been tagged with an enzyme, fluorescent molecule or other detectable molecule (for example, biotin, which binds to avidin or streptavidin, or a hemin molecule, which binds to the apoenzyme portion of catalase).

In a preferred embodiment, both the functional tail R and the additional moiety are dyes. One or both of the tail and additional moiety dyes can be fluorescent dyes. Preferably, one of the dyes is fluorescent. In one preferred embodiment the functional tail comprises a fluorescent dye and the additional moiety comprises a quencher. The fluorescent dye and quencher molecule operate together such that the fluorescence of the dye is repressed when the dye is bound to the AP site probe, but the fluorescence of the dye is detectable when the phosphodiester bond between the NA and tail R is hydrolyzed or cleaved by the enzyme. This fluorescence detection strategy is known as Fluorescence Resonance Energy Transfer (FRET). According to a FRET technique, one of the dyes servers as a reporter dye and the other dye is a quencher that substantially decreases or eliminates fluorescence of the reporter dye when both of the dyes are bound to the same molecule in proximity of each other. The fluorescence of the reporter dye is detected when released from the proximity of the quencher dye. Cleavage of the AP site probe functional tail releases the reporter dye from its quencher counterpart allowing for a detectable increase in the reporter fluorescence and detection of the target nucleic acids. The quenching dye can be a fluorescent dye or non-fluorescent dye (dark quencher). See, U.S. Patent Publication Nos. 2003/0113765 and 2003/0096254 and PCT Publication No. WO 01/42505 for fluorophore and quencher examples, both of which are hereby incorporated herein by reference.

The present invention includes a composition comprising a solid support and an AP site probe immobilized thereon. In such a case, one of the moieties conjugated to the probe can be a moiety that serves to attach the probe to the solid support. This moiety or solid support linker can be attached anywhere within or be a structural part of the NA and functional tail R structures of the probe of the present invention. In one embodiment, the AP site probe is covalently attached to a solid support through a Schiff base type linkage, as described in U.S. Pat. No. 6,548,652, incorporated herein by reference. In assays of the present invention, an AP site probe is typically included at concentrations of about 50-200 nM, more typically at concentrations of about 100-175 nM, and preferably at concentrations of about 150 nM. One of skill in the art will appreciate that the probe concentrations provided above can be altered depending on a variety of factors, including the amount of target, as well as the characteristics of the dye or quencher used.

An enhancer is an oligo- or polynucleotide designed to form a duplex with the target nucleic acid positioned immediately 5′- to the target-AP site probe. The combined, probe-enhancer-target complex simulates a naturally occurring nucleic acid atypical abasic site that is recognized by cellular exo- and endonuclease repair enzymes. Although the tail R cleavage reaction can be achieved without the enhancer, the presence of an enhancer generally improves the kinetics the reaction. The probe and enhancer form duplexes with the target nucleic acid that are positioned next to each other leaving one, non-paired base of the target between the duplexes. Although this is a preferred design, cleavage of the tail R in the target-probe complex can be achieved in absence of the enhancer, or when the number of non-paired, target polynucleotide bases between two duplexes shown is 1, 2, 3, 4, 5 or more bases.

The structural requirements and limitations for an enhancer are essentially the same as for a NA component of an AP site probe, described above. Generally, the number of nucleotides in an enhancer oligonucleotide can range from 3 to 50, 100 or 200 nucleotides in length. Usually, the length of an enhancer is from 5 to 30 nucleotides. More typically, the length of the enhancer is 6-25, 7-20, or 8-15 nucleic acids. Most often, an enhancer component is about 10, 12, 14, 16, 18 or 20 nucleic acids in length. Usually, an enhancer oligonucleotide component will have a hybridization melting temperature of about 10 to 80° C., more typically of about 20 to 70° C., and preferably about 30° C., 40° C., 50° C., 60° C. or 70° C. An enhancer oligonucleotide will usually have a comparatively equal or higher hybridization melting temperature in comparison to the melting temperature of the NA component of the AP site probe. Usually, the melting temperature will be about 5 to 30° C., more typically about 10 to 20° C., and preferably about 8° C., 10° C., 15° C., or 20° C. higher than the melting temperature of the NA component of the AP site probe.

Preferably, the enhancer is DNA. An oligo- or polydeoxyribonucleotide enhancer is useful for detecting DNA and RNA target nucleic acids. The enhancer can also be RNA. In another embodiment, an enhancer can contain both DNA and RNA. Preferably, DNA bases are located at the 5′-end of the enhancer while RNA bases are at its 3′-end. Preferably, at least the four 5′-terminal bases of the enhancer are DNA bases.

In another embodiment, the enhancer contains nucleotides with modified, synthetic or unnatural bases, including any modification to the base, sugar or backbone. Preferably, modified bases increase thermal stability of the enhancer-target duplex in comparison to enhancer sequences that contain only natural bases. Specific modified bases are the same as those described for a probe.

In another embodiment, some or all nucleotides of the enhancer are substituted or contain independently different sugar-phosphate backbone modifications, including, 2′-O-alkyl RNA nucleotide, phosphorotioate internucleotide linkage, PNA (peptide nucleic acid), LNA (locked nucleic acid). References describing these and other potentially useful sugar-phosphate backbone modifications are provided above.

The enhancer optionally can contain some functional tails or markers conjugated to either end of the enhancer or in the middle of it. These moieties should not interfere with the template-specific cleavage of the probe R tail. In a preferred embodiment, these moieties are attached to the 3′-end of the enhancer. In another preferred embodiment, these moieties are conjugated to nucleotide bases of the enhancer such that, when the enhancer-target duplex is formed, the moieties are located within the major groove of this duplex. Enhancer moieties can serve to improve the enhancer hybridization properties. Examples of such moieties include minor groove binders and intercalators.

The present invention also encompasses a composition comprising an enhancer immobilized on a solid support. A moiety conjugated to the enhancer can serve to attach the enhancer to the solid support. This moiety or solid support linker can be attached anywhere within or be a structural part of the enhancer.

Modifications of the bases and sugar-phosphate backbone as well as other functional moieties conjugated to the enhancer can serve to improve the sequence specificity of target-enhancer duplex formation resulting in increased thermodynamic differences in binding between the enhancer and a matched target nucleic acid in comparison to binding between the enhancer and a mismatched target nucleic acid.

In assays of the present invention, an enhancer, when included, is typically added at concentrations of about 50-200 nM, more typically at concentrations of about 100-175 nM, and preferably at concentrations of about 150 nM. One of skill in the art will appreciate that the enhancer concentrations provided above can be altered depending on a variety of factors, including the amount of target, as well as the amount of probe used and its characteristics.

An enzyme used in conjunction with the AP site probe is an endonuclease or exonuclease that recognizes an Apurinic/Apyrimidinic (AP) site or atypical AP site moiety simulated by an AP site probe duplexed with a target nucleic acid complex, and preferentially hydrolyzes or cleaves the phosphodiester bond between the probe and the functional tail R. An enhancer can be used to increase the kinetics of the tail-cleavage reaction. An enzyme useful in the present methods preferentially does not cleave the NA part of the probe or the target nucleic acid. Otherwise, enzymes which cleave the probe NA or target nucleic acid at an efficiency that is substantially lower than target-specific tail cleavage can still find use in practicing the present methods. To minimize non-specific detection of the target nucleic acid, the enzyme preferentially does not cleave the tail R of the probe in absence of the target nucleic acid.

In a preferred embodiment, the enzyme is an AP endonuclease. The enzyme can be a class I or a class II AP endonuclease. Preferably, the enzyme is a class II endonuclease. Enzymes that belong to this family are isolated from variety of organisms, and any class II endonuclease that specifically recognizes an AP abasic site and specifically hydrolyzes the phosphodiester backbone on the 5′ side of the AP site can be used in the present methods. Exemplified class II AP endonucleases include Endonuclease IV and Exonuclease III from E. coli, human APE1/REF-1 endonuclease, yeast APN1 endonuclease, exonuclease III homologous enzymes from Drosophila (Rrpl) and Arabidopsis (Arp) and thermostable endonuclease IV from Thermotoga maritima. Other AP endonucleases useful for detection and/or amplification systems requiring an AP site probe can be identified through the National Center for Biotechnological Information Entrez/PubMed nucleotide and protein databases accessed through the website www.ncbi.nlm.nih.gov/. Enzymes homologous in structure and function to the E. coli Exonuclease III family of AP nucleases are also of use in the present invention (Mol, et al., Mutat. Res. 460:211 (2000); Ramotar, Biochem. Cell Bio. 75:327 (1997)). The structure and function of apurinic/apyrimidinic endonucleases is reviewed by Barzilay and Hickson in Bioessays 17:713 (1995).

In a preferred embodiment, the enzyme is an E. coli Endonuclease IV. An E. coli Endonuclease IV exhibits catalytic activity between room temperature (25° C.) and 75° C., preferably between 40-70° C. or 40-60° C., and more preferably between 60-70° C. or 65-75° C. The temperature of a target nucleic acid detection assay is preferably determined by the hybridization melting temperature of an AP site probe, where the temperature of the reaction conditions is preferably within 5, 4, 3, 2, 1 or 0 degrees, above or below, of the probe melting temperature, Tₘ. Optimum catalytic activity of an Endonuclease IV is observed within a pH range of 7.5-9.5, preferably between pH 8.0-9.0, most preferably at about pH 8.5-9.0. An abasic site assay using an Endonuclease IV enzyme is preferably carried out using a buffer that maintains a steady pH value of between 7.5-9.5 over varying temperatures. Preferred buffers include HEPPS (4-(2-hydroxyethyl)-1-piperazinpropan-sulfonic acid) and HEPES (4-(2-Hydroxyethyl)piperazine-1-ethanesulfonic acid). In a preferred embodiment, the buffer used is HEPPS-KOH. In certain embodiments, a TRIS buffer is also appropriate. Additional biological buffers of potential use can be found through Sigma-Aldrich (St. Louis, Mo., www.sigma.com). Usually, the reaction conditions contain enzyme in nanomolar concentrations, but tail cleaving activity can be observed when the enzyme is provided in picomolar concentrations, and in certain cases in femtomolar concentrations.

Either part of the endonuclease tail-cleavage reaction, the NA containing part or the tail R containing part or alternatively both of them independently, can be detected. Suitable reporter groups for attaching to the functional tail R include beads, nanoparticles (Taton, et al., Science 289:1757 (2000), chemiluminescers, isotopes, enzymes and fluorophores. A variety of physical or chemical methods can be used for detection of the cleavage product. Depending on the nature of the markers used, these methods include, for example, chromatography and electron-, UV-, IR-, mass-, radio-, fluorescence spectroscopy including fluorescence polarization and the like.

In a preferred embodiment, cleavage of the functional tail R comprises a fluorophore reporter group and is detected by fluorescence spectroscopy. Suitable fluorophores include the resorufin dyes, coumarin dyes, xanthene dyes, cyanine dyes, BODIPY dyes and pyrenes. Preferably, the functional tail R comprises a fluorescent dye with a xanthene core structure. Additional fluorophores appropriate for incorporation into the functional tail R are described in PCT Publication Nos. WO 01/142505 and WO 06/020947 and in Haugland, Handbook of Fluorescent Probes and Research Products, Ninth Ed., (2002), published by Molecular Probes, Eugene, Oreg. (accessible at www.probes.com/handbook/).

In some embodiments, background fluorescence of a fluorophore incorporated on the functional tail R, is minimized by attaching a quencher to the AP site probe. Typically, a quenching molecule is covalently attached to the 5′ end of the probe through a linker that is not cleaved by an enzyme. In some embodiments, a quencher is linked to the middle or the 3′ end of the probe. When a quencher is attached to the 3′ end of the probe, it is usually incorporated into the functional tail R as a “cleavable quencher,” and the fluorophore is then attached to the middle or the 5′ end of the probe. However, any molecule that neutralizes or masks the fluorescence of a fluorophore incorporated in an uncleaved functional tail R finds use as a quencher in the present invention. Other quencher molecules suitable to attach to an AP site probe and guidance for selecting appropriate quencher and fluorophore pairs is provided in Haugland, supra. Additional guidance is provided in U.S. Pat. Nos. 3,996,345 and 4,351,760, and U.S. Publication Nos. 2003/0096254 and 2003/0113765 and in co-owned U.S. patent application Ser. No. 09/457,616, filed on Dec. 8, 1999, each of which is hereby incorporated herein by reference.

Fluorophore and cleavable quencher molecules are typically attached to an AP site probe through a linker that is specifically cleaved by an enzyme. A linker can be rigid or flexible. Preferably the linker structurally mimics a naturally occurring abasic site, and is cleaved by an Endonuclease IV. Preferably the C1 carbon of the linker, attached to the phosphate, is a primary carbon. Preferably the linker comprises a phosphate. Suitable commercially available chemical linkers can be purchased through Pierce Biotechnology, Rockford, Ill. and Molecular Probes, Eugene, Oreg. Suitable methods for attaching reporter groups such as fluorophores and quenchers through linkers to oligonucleotides are described in, for example, U.S. Pat. Nos. 5,512,677; 5,419,966; 5,696,251; 5,585,481; 5,942,610 and 5,736,626, each of which are hereby incorporated herein by reference.

In a preferred embodiment the linker is a rigid linker. In one preferred embodiment, the rigid linker is a hydroxyprolinol linker. Hydroxyprolinol linkages are described in U.S. Pat. Nos. 5,419,966; 5,512,677; 5,519,134; and 5,574,142 each of which is incorporated herein by reference. Cleavage of the functional tail R attached through a rigid linker, i.e., a hydroxyprolinol linker, requires greater concentrations of enzyme and exhibits decreased catalytic rates, but is highly specific. Generally, the Endonuclease IV enzyme does not detectably cleave functional tails R attached to an AP site probe through a rigid linker, such as a hydroxyprolinol linker, in the absence of a target nucleic acid.

In some embodiments, it is desirable to attach the functional tail R through a flexible linker. Cleavage of the functional tail R is more efficient when attached through a flexible linker, however, decreased specificity is observed because detectable tail-cleavage occurs in the absence of a target nucleic acid. Non-specific cleavage of functional tails R attached through a flexible linker can be minimized by adding a competitive binding substrate that is more favorable to the enzyme than an unduplexed probe but less favorable than the probe duplexed with a target nucleic acid, i.e., a “decoy.” In one embodiment unmelted genomic DNA is added to the reaction as a decoy to minimize cleavage of the AP site probe functional tail R in the absence of a target nucleic acid.

The ability of particular tail structures to serve as specific substrates of an AP endonuclease can be determined using an assay that provides a probe/target nucleic acid/enhancer complex as a single hairpin structure. Preferably the hairpin structure has one unpaired nucleic acid, thereby simulating a naturally occurring abasic site residing in duplexed nucleic acids. In other embodiments, the test assay hairpin structure can have zero or two unpaired nucleic acids. In such a test assay, the cleavage of the functional tail R is detected by measuring the release of the reporter group attached to a hairpin structure in comparison to release of the reporter group attached to an unduplexed AP site probe. A tail structure that serves as a specific substrate for an AP endonuclease will be cleaved from a hairpin structure at a faster catalytic rate in comparison to its cleavage rate from an unduplexed AP site probe. A tail structure that serves as a specific substrate preferably exhibits a ratio of specific cleavage, in the presence of the hairpin structure, to non-specific cleavage, in the presence of an unduplexed AP site probe, of at least 50-, 75-, or 100-fold, more preferably of 300-, 400-, 500-, 600-, 700-, 800-, 900- or 1000-fold, and can exhibit ratios of greater than 1000-fold, as measured by the reporter group signal (i.e., Fluorescence Units per minute of a fluorophore reporter group). In some embodiments, the hairpin substrate design does not incorporate a quencher moiety. Nevertheless AP endonuclease cleavage of the fluorescent tail increases the dye fluorescence by approximately two times. The fluorescent signal outcome of the assay can be improved by incorporation of a quenching moiety within the hairpin sequence that represents an enhancer. Those skilled in the art will appreciate that the hairpin substrate can be used for detection as well as for quantitative measurement of AP endonuclease activity in different media.

In other embodiments, the NA part of the AP site probe is detected. For instance, the products of the probe tail-cleavage reaction can be detected as a result of another reaction that follows the cleavage reaction or occurs simultaneously with it. Cleavage of the tail R from the probe generates a “free” 3′-hydroxyl group that can be, for example, extended by a polymerase in a template-dependent polynucleotide synthesis in the presence of NTPs such that the tail-OFF probe would serve as a primer complexed with template. In some embodiments, the strands of a probe extension nucleotide synthesis are the detectable reaction product. Some NTPs incorporated in a probe extension can optionally carry a detectable marker. Incorporation of one or more detectable markers into a probe extension product simplifies the detection of the synthesized nucleotide strands.

In one embodiment, a probe is linked to an enhancer so as these two components of the reaction complex are associated with each other during the tail cleaving reaction. The linker can be a covalent or a non-covalent linker, i.e., when interaction between a probe and enhancer is provided by hydrogen bonds or Van der Waals forces. A probe-enhancer linker can be attached at any position within the probe and enhancer. Preferably, the linker does not block the tail cleaving reaction, and is of an appropriate length to support the tail cleaving reaction. Further, a linker useful in a tail cleaving assay will not compromise the ability of the AP site probe or enhancer to form duplexes with a target nucleic acid. Finally, a preferred linker is not cleaved by an AP endonuclease. When attached through a linker, the probe and enhancer are components of one molecule or complex. Linked probe-enhancer molecules or complexes can be immobilized on a solid support.

In preferred embodiments, a probe-enhancer linker is comprised of individual or combined repeats of substituted alkyl backbone moieties, including (—OCH₂CH₂—)ₙ, (—OCH₂CH₂—OPO₂—)ₙ or —O(CH₂)ₙO—. Typically, n is from 1-100, more typically n is 10, 20, 40, 50, 60 or 80. In other embodiments, a linker is a flexible polypeptide chain, for instance, dihydropyrroloindole peptides or a series of one or more repeats of a Gly-(Ser)₄ polypeptide sequence. In another embodiment, the linker is an oligonucleotide, such as poly A or poly T and the like. In yet another embodiment, the linker is an alkyl chain having a backbone typically of about 100, 200 or 300 atoms, more typically of about 40, 60 or 80 atoms. Other alkyl linkers of potential use are described in U.S. Patent Publication No. 2003/0113765, incorporated herein by reference. Additional linkers that may find use are described by Dempey, et al., Nucleic Acids Res. 27:2931 (1999); Lukhtanov, et al., Nucleic Acids Res. 25:5077 (1997); Lukhtanov, et al., Bioconjug. Chem. 7:564 (1996); and Lukhtanov, et al., Bioconjug. Chem. 6:418 (1995). Appropriate linkers can be obtained from commercially available sources, for example from Pierce Biotechnology, Rockford Ill. Guidance for selecting an appropriate linker for attaching oligonucleotides is provided in Haugland, Handbook of Fluorescent Probes and Research Products, supra. These linkers also find application in attaching an AP site probe or an enhancer to a solid support.

E. Oligonucleotides and Modified Oligonucleotides

The terms oligonucleotide, polynucleotide and nucleic acid are used interchangeably to refer to single- or double-stranded polymers of DNA or RNA (or both) including polymers containing modified or non-naturally-occurring nucleotides, or to any other type of polymer capable of stable base-pairing to DNA or RNA including, but not limited to, peptide nucleic acids which are disclosed by Nielsen et al. Science 254:1497-1500 (1991); bicyclo DNA oligomers (Bolli et al., Nucleic Acids Res. 24:4660-4667 (1996)) and related structures.

The oligonucleotides of the present invention are generally prepared using solid phase methods known to those of skill in the art. In general, the starting materials are commercially available, or can be prepared in a straightforward manner from commercially available starting materials, using suitable functional group manipulations as described in, for example, March, et al., ADVANCED ORGANIC CHEMISTRY—Reactions, Mechanisms and Structures, 4th ed., John Wiley & Sons, New York, N.Y., (1992).

The oligonucleotides of the invention can comprise any naturally occurring nucleotides, non-naturally occurring nucleotides, or modified nucleotides known in the art.

The oligonucleotide primers and probes of the present invention can include the substitution of one or more naturally occurring nucleotide bases within the oligomer with one or more non-naturally occurring nucleotide bases or modified nucleotide bases so long as the primer can initiate amplification of a target nucleic acid sequence in the presence of a polymerase enzyme.

For example, the oligonucleotide primers may also comprise one or more modified bases, in addition to the naturally-occurring bases adenine, cytosine, guanine, thymine and uracil. Modified bases are considered to be those that differ from the naturally-occurring bases by addition or deletion of one or more functional groups, differences in the heterocyclic ring structure (i.e., substitution of carbon for a heteroatom, or vice versa), and/or attachment of one or more linker arm structures to the base. Preferred modified nucleotides are those based on a pyrimidine structure or a purine structure, for example, 7 deazapurines and their derivatives and pyrazolopyrimidines (described in, for example, WO 90/14353 and U.S. Pat. No. 6,127,121).

Exemplified modified bases for use in the present invention include the guanine analogue 6 amino-1H-pyrazolo[3,4-d]pyrimidin-4(5H)-one (ppG or PPG, also Super G) and the adenine analogue 4-amino-1H-pyrazolo[3,4-d]pyrimidine (ppA or PPA). The xanthene analogue 1H-pyrazolo[5,4-d]pyrimidin-4(5H)-6(7H)-dione (ppX) can also be used. These base analogues, when present in an oligonucleotide, strengthen hybridization and improve mismatch discrimination. All tautomeric forms of naturally-occurring bases, modified bases and base analogues may be included in the oligonucleotide conjugates of the invention. Other modified bases useful in the present invention include 6-amino-3-prop-1-ynyl-5-hydropyrazolo[3,4-d]pyrimidine-4-one, PPPG; 6-amino-3-(3-hydroxyprop-1-yny)1-5-hydropyrazolo[3,4-d]pyrimidine-4-one, HOPPPG; 6-amino-3-(3-aminoprop-1-ynyl)-5-hydropyrazolo[3,4-d]pyrimidine-4-one, NH₂PPPG; 4-amino-3-(prop-1-ynyl)pyrazolo[3,4-d]pyrimidine, PPPA; 4-amino-3-(3-hydroxyprop-1-ynyl)pyrazolo[3,4-d]pyrimidine, HOPPPA; 4-amino-3-(3-aminoprop-1-ynyl)pyrazolo[3,4-d]pyrimidine, NH₂PPPA; 3-prop-1-ynylpyrazolo[3,4-d]pyrimidine-4,6-diamino, (NH₂)₂PPPA; 2-(4,6-diaminopyrazolo[3,4-d]pyrimidin-3-yl)ethyn-1-ol, (NH₂)₂PPPAOH; 3-(2-aminoethynyl)pyrazolo[3,4-d]pyrimidine-4,6-diamine, (NH₂)₂PPPANH₂; 5-prop-1-ynyl-1,3-dihydropyrimidine-2,4-dione, PU; 5-(3-hydroxyprop-1-ynyl)-1,3-dihydropyrimidine-2,4-dione, HOPU; 6-amino-5-prop-1-ynyl-3-dihydropyrimidine-2-one, PC; 6-amino-5-(3-hydroxyprop-1-yny)-1,3-dihydropyrimidine-2-one, HOPC; and 6-amino-5-(3-aminoprop-1-yny)-1,3-dihydropyrimidine-2-one, NH₂PC; 5-[4-amino-3-(3-methoxyprop-1-ynyl)pyrazol[3,4-d]pyrimidinyl]-2-(hydroxymethyl)oxolan-3-ol, CH₃ OPPPA; 6-amino-1-[4-hydroxy-5-(hydroxymethyl)oxolan-2-yl]-3-(3-methoxyprop-1-ynyl)-5-hydropyrazolo[3,4-d]pyrimidin-4-one, CH₃OPPPG; 4,(4,6-Diamino-1H-pyrazolo[3,4-d]pyrimidin-3-yl)-but-3-yn-1-ol, Super A; 6-Amino-3-(4-hydroxy-but-1-ynyl)-1,5-dihydro-pyrazolo[3,4-d]pyrimidin-4-one; 5-(4-hydroxy-but-1-ynyl)-1H-pyrimidine-2,4-dione, Super T; 3-iodo-1H-pyrazolo[3,4-d]pyrimidine-4,6-diamine((NH₂)₂PPAI); 3-bromo-1H-pyrazolo[3,4-d]pyrimidine-4,6-diamine((NH₂)₂PPABr); 3-chloro-1H-pyrazolo[3,4-d]pyrimidine-4,6-diamine ((NH₂)₂PPACl); 3-Iodo-1H-pyrazolo[3,4-d]pyrimidin-4-ylamine (PPAI); 3-Bromo-1H-pyrazolo[3,4-d]pyrimidin-4-ylamine(PPABr); and 3-chloro-1H-pyrazolo[3,4-d]pyrimidin-4-ylamine(PPACl).

In addition to the modified bases noted above, the oligonucleotides of the invention can have a backbone of sugar or glycosidic moieties, preferably 2-deoxyribofuranosides wherein all internucleotide linkages are the naturally occurring phosphodiester linkages. In alternative embodiments however, the 2-deoxy-β-D-ribofuranose groups are replaced with other sugars, for example, β-D-ribofuranose. In addition, β-D-ribofuranose may be present wherein the 2-OH of the ribose moiety is alkylated with a C₁₋₆ alkyl group (2-(O—C₁₋₆ alkyl) ribose) or with a C₂₋₆ alkenyl group (2-(O—C₂₋₆ alkenyl) ribose), or is replaced by a fluoro group (2-fluororibose). Related oligomer-forming sugars useful in the present invention are those that are “locked”, i.e., contain a methylene bridge between C-4′ and an oxygen atom at C-2′. Other sugar moieties compatible with hybridization of the oligonucleotide can also be used, and are known to those of skill in the art, including, but not limited to, α-D-arabinofuranosides, α-2′-deoxyribofuranosides or 2′,3′-dideoxy-3′-aminoribofuranosides. Oligonucleotides containing α-D-arabinofuranosides can be prepared as described in U.S. Pat. No. 5,177,196. Oligonucleotides containing 2′,3′-dideoxy-3′-aminoribofuranosides are described in Chen et al. Nucleic Acids Res. 23:2661-2668 (1995). Synthetic procedures for locked nucleic acids (Singh et al, Chem, Comm., 455-456 (1998); Wengel J., Acc. Chem. Res., 32:301-310 (1998)) and oligonucleotides containing 2′-halogen-2′-deoxyribofuranosides (Palissa et al., Z. Chem., 27:216 (1987)) have also been described. The phosphate backbone of the modified oligonucleotides described herein can also be modified so that the oligonucleotides contain phosphorothioate linkages and/or methylphosphonates and/or phosphoroamidates (Chen et al., Nucl. Acids Res., 23:2662-2668 (1995)). Combinations of oligonucleotide linkages are also within the scope of the present invention. Still other backbone modifications are known to those of skill in the art.

The ability to design probes and primers in a predictable manner using an algorithm, that can direct the use or incorporation of modified bases, minor groove binders, fluorophores and/or quenchers, based on their thermodynamic properties have been described in co pending application Ser. No. 10/032,307, filed Dec. 21, 2001. Accordingly, the use of any combination of normal bases, unsubstituted pyrazolo[3,4-d]pyrimidine bases (e.g., PPG and PPA), 3-substituted pyrazolo[3,4-d]pyrimidines, modified purine, modified pyrimidine, 5-substituted pyrimidines, universal bases, sugar modification, backbone modification or a minor groove binder to balance the Tₘ (e.g., within about 5-8° C.) of a hybridized product with a modified nucleic acid is contemplated by the present invention.

F. Quenchers

Recently developed detection methods employ the process of fluorescence resonance energy transfer (FRET) for the detection of probe hybridization rather than direct detection of fluorescence intensity. In this type of assay, FRET occurs between a donor fluorophore (reporter) and an acceptor molecule (quencher) when the absorption spectrum of the quencher molecule overlaps with the emission spectrum of the donor fluorophore and the two molecules are in close proximity. The excited-state energy of the donor fluorophore is transferred to the neighboring acceptor by a resonance dipole-induced dipole interaction, which results in quenching of the donor fluorescence. If the acceptor molecule is a fluorophore, its fluorescence may sometimes be increased. The efficiency of the energy transfer between the donor and acceptor molecules is highly dependent on distance between the molecules. Equations describing this relationship are known. The Forster distance (Rₒ) is described as the distance between the donor and acceptor molecules where the energy transfer is 50% efficient. Other mechanisms of fluorescence quenching are also known, such as, collisional and charge transfer quenching. There is extensive guidance in the art for selecting quencher and fluorophore pairs and their attachment to oligonucleotides (Haugland, R. P., HANDBOOK OF FLUORESCENT PROBES AND RESEARCH CHEMICALS, Sixth Edition, Molecular Probes, Eugene, Oreg., 1996; U.S. Pat. Nos. 3,996,345 and 4,351,760 and the like). Preferred quenchers are described in co-owned U.S. Pat. Nos. 6,727,356 and 6,790,945 and incorporated herein by reference. Additional structures (e.g., mono- and bis-azo dyes) with different combinations of substituents at various positions can be prepared based on compounds and methods known in the dye chemistry field (summarized in the Color Index, Issue 3 on CDD-ROM, pages 4009-4324; Society of Dyers and Colourists, Bradford, England; http://www.sdc.org.uk; and see also WO 01/86001).

The quenchers disclosed above cover the range from about 400-800 nm, and many demonstrate improved quenching when attached to a MGB. While the modified versions illustrate —N(CH₂CH₂OH)₂ as a preferred linking group to be used to couple the quencher to oligonucleotides, MGB or solid support, examples of other suitable linkers are known in the art or are provided herein.

Preferred quenchers for each of the aspects of the invention herein are selected from those disclosed above, as well as bis azo quenchers from Biosearch Technologies, Inc. (provided as Black Hole™ Quenchers: BH-1, BH-2 and BH-3), Dabcyl, TAMRA and carboxytetramethyl rhodamine

G. Fluorophores

Fluorophores useful in the present invention are generally fluorescent organic dyes that have been derivatized for attachment to the terminal 5′ carbon of the oligonucleotide probe, preferably via a linking group. One of skill in the art will appreciate that suitable fluorophores are selected in combination with a quencher which is typically also an organic dye, which may or may not be fluorescent.

There is a great deal of practical guidance available in the literature for selecting appropriate fluorophore-quencher pairs for particular probes. See, for example, Clegg (cited above); Wu et al. (cited above); Pesce et al., editors, FLUORESCENCE SPECTROSCOPY (Marcel Dekker, New York, 1971); White et al., FLUORESCENCE ANALYSIS: A PRACTICAL APPROACH (Marcel Dekker, New York, 1970); and the like. The literature also includes references providing exhaustive lists of fluorescent and chromogenic (quenching) molecules and their relevant optical properties for choosing fluorophore-quencher pairs, e.g., Berlman, HANDBOOK OF FLUORESCENCE SPECTRA OF AROMATIC MOLECULES, 2ND EDITION (Academic Press, New York, 1971); Griffiths, COLOUR AND CONSTITUTION OF ORGANIC MOLECULES (Academic Press, New York, 1976); Bishop, editor, INDICATORS (Pergamon Press, Oxford, 1972); Haugland, HANDBOOK OF FLUORESCENT PROBES AND RESEARCH CHEMICALS (Molecular Probes, Eugene, 1992); Pringsheim, FLUORESCENCE AND PHOSPHORESCENCE (Interscience Publishers, New York, 1949); and the like. Additionally, methods for derivatizing fluorophores and quenchers for covalent attachment via common reactive groups are also well known. See, for example, Haugland (cited above); Ullman et al., U.S. Pat. No. 3,996,345; Khanna et al., U.S. Pat. No. 4,351,760; and the like.

Phosphonylated dyes disclosed in co-owned U.S. patent application Ser. No. 11/202,635 are particularly preferred and includes xanthene-, cyanine-, coumarin-, phenoxazine-, Bodipy-based fluorophores. Other preferred fluorophores are those based on xanthene dyes, a variety of which are available commercially with substituents useful for attachment of either a linking group or for direct attachment to an oligonucleotide. Another group of fluorescent compounds are the naphthylamines, having an amino group in the α- or β-position. Included among such naphthylamino compounds are 1-dimethylaminonaphthyl-5-sulfonate, 1-anilino-8-naphthalene sulfonate and 2-ρ-toluidinyl-6-naphthalene sulfonate. Other dyes include 3-phenyl-7-isocyanatocoumarin, acridines, such as 9-isothiocyanatoacridine and acridine orange; N-(p-(2-benzoxazolyl)phenyl)maleimide; benzoxadiazoles, stilbenes, pyrenes, and the like. Still other suitable fluorophores include the resorufin dyes, rhodamine dyes, cyanine dyes and BODIPY dyes.

These dyes and appropriate linking methodologies for attachment to oligonucleotides are described in many references, e.g., Khanna et al. (cited above); Marshall, Histochemical J., 7:299-303 (1975); Menchen et al., U.S. Pat. No. 5,188,934; Menchen et al., European Patent Application 87310256.0; and Bergot et al., International Application No. PCT/US90/05565.

More particularly, the fluorophores described herein can be attached to the oligonucleotide portions using, for example, chemical or enzymatic methods. By way of example, methods for incorporation of reactive chemical groups into oligonucleotides, at specific sites, are well-known to those of skill in the art. Oligonucleotides containing a reactive chemical group, located at a specific site, can be combined with a label attached to a complementary reactive group (e.g., an oligonucleotide containing a nucleophilic reactive group can be reacted with a label attached to an electrophilic reactive group) to couple a label to a probe by chemical techniques. Exemplary labels and methods for attachment of a label to an oligonucleotide are described, for example, in U.S. Pat. No. 5,824,796; U.S. Pat. No. 5,210,015; Kessler (ed.), Nonradioactive Labeling and Detection of Biomolecules, Springer-Verlag, Berlin, 1992; Kricka (ed.) Nonisotopic DNA Probe Techniques, Academic Press, San Diego, 1992; Howard (ed.) Methods in Nonradioactive Detection, Appleton & Lange, Norwalk, 1993. Non-specific chemical labeling of an oligonucleotide can be achieved by combining the oligonucleotide with a chemical that reacts, for example, with a particular functional group of a nucleotide base, and simultaneously or subsequently reacting the oligonucleotide with a label. See, for example, Draper et al. (1980) Biochemistry 19:1774-1781. Enzymatic incorporation of label into an oligonucleotide can be achieved by conducting enzymatic modification or polymerization of an oligonucleotide using labeled precursors, or by enzymatically adding label to an already-existing oligonucleotide. See, for example, U.S. Pat. No. 5,449,767. Examples of modifying enzymes include, but are not limited to, DNA polymerases, reverse transcriptases, RNA polymerases, etc. Examples of enzymes which are able to add a label to an already-existing oligonucleotide include, but are not limited to, kinases, terminal transferases, ligases, glycosylases, etc.

For each of the aspects of the present invention, preferred fluorophores are selected from xanthenes, cyanines, BODIPY analogs, 5-FAM, 6-FAM, TET™, JOE™, HEX™, VIC™, NED™, TAMA™, ROX™, Bothell Blue™, Gig Harbor Green™ and Yakima Yellow™. These fluorophores are generally available from commercial sources such as Applied Biosystems Inc., Foster City, Calif. and Epoch Biosciences, Inc., Bothell, Wash.

### IV. Kits

The invention further provides kits comprising components for carrying out the methods described herein. For example, a kit may comprise one container that holds a nicking enzyme (e.g., N.BbvCI), another container that holds an extension primer, another container that holds a bumper primer, another container that holds an extension primer, another container that holds an AP site probe, another container that holds an AP endonuclease, another container that holds an enhancer, and combinations of thereof.

### EXAMPLES

The following examples are provided to illustrate, but to limit the presently claimed invention.

### Example 1

This example demonstrates the isothermal generation of ssDNA by strand displacement utilizing a nicking enzyme and a polymerase in a SDA amplification reaction and fluorescent detection of amplified target with endonuclease IV signal detection system in a homogenous reaction.

Assay Design and Oligonucleotide Component Structures for Mycobacterium tuberculosis Detection

A fragment of the Mycobacterium tuberculosis IS6110 sequence (GenBank X52471) is shown in Sequence 1. The endogenous N.BbvC1B recognition site is shown in bold and underlined. The locations of the complementary target specific sequences of the amplification primers are shown in bold italics, while the locations of the bumper sequences are shown in lower case. The probe sequence for the endonuclease IV assay is underlined.

Sequence 1.
GAGACCTCAGCCGGCGGCTGGTCTCTGGCGTTGAGCGTAGTAGGCAGCCTCGAGTTCGACCG
GCGGGACGTCGCCGCAGTACTGGTAGAGGCGGCGATGGTTGAACCAGTCGACCCAGCGCGCG
GTGGCCAACTCGACATCCTCGATGGACCGCCAGGGCTTGCCGGGTTTGATCAGCTCGGTCTT
GTATAGGCCGTTGATCGTCTCGGCTAGTGCATTGTCATAGGAGCTTCCGACCGCTCCGACCG
ACGGTTGGATGCCTGCCTCGGCGAGCCGCTcgctgaaccggatCGATGTGT [symbol] [symbol]
ATCCGTATGGTGGATAACGTCTTTCA [symbol] GCCTTCTTgttggcgggtccaGAT
GGCTTGCTCGATCGCGTCGAGGACCATGGAGGTGGCCATCGTGGAAGCGACCCGCCAG

### Oligonucleotide Sequences

Sequence 2, Forward primer:
GCATTATAGTACCTGTCTCCTCAGC [symbol]
Sequence 3, Reverse primer:
TTGAATAGTCGGTTACTTCCTCAGC [symbol]
Sequence 4, Forward bumper:
cgctgaaccggat
Sequence 5, Reverse bumper:
tggacccgccaac
Sequence 6, Probe:
Q - TCCGTA*TGGTG - F1

where Q is a quencher and in this example the Eclipse Dark Quencher and Fl is a fluorophore and in this example Gig Harbor Green™ fluorescent dye. A* is Super A™ modified base.

### DNA Sample

Genomic DNA from M. tuberculosis strain SBRI10.

### Homogeneous Isothermal SDA

Amplifications were performed on samples containing M. tuberculosis target DNA (from strain SBR110) in 10 μl final volume in a Rotor-Gene 3000 thermocycler (Corbett Research). Each sample contained 36 mM K₂HPO₄, pH7.6, 3.75 mM MgCl₂, 0.25 mM each dNTPs (dATP, dGTP, dCTP and TTP), 10 ng of human genomic DNA, 50 nM forward primer, 500 nM reverse primer, 50 nM each bumper, 4U Bst DNA polymerase, 4U BbvC1B (New England Biolabs), 0.1U Endo IV (Trevigen) diluted in Diluent A (New England Biolabs). After addition of all above components, including the three enzymes at room temperature, reaction tubes were placed directly in the thermocycler and were incubated at 49° C. for 50 min. Fluorescent readings were taken at one minute intervals in the FAM channel with an excitation and emission wavelengths of 470 and 510 nm, respectively.

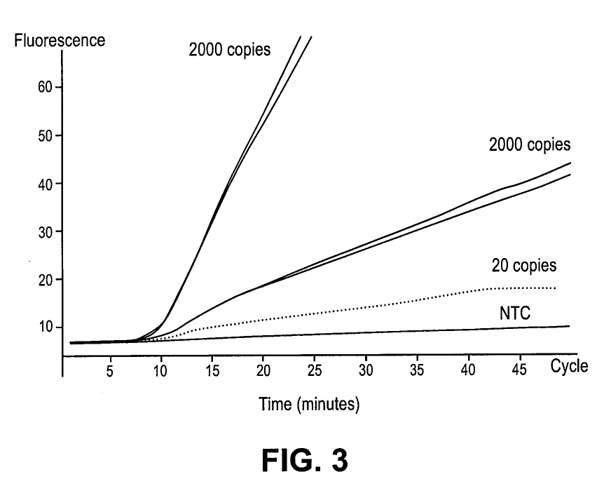

Amplification of no-template control, 20, 200 and 2000 copies of target is shown in FIG. 3. As shown amplification occurs rapidly and 20 copies could be determined within 30 minutes.

### Example 2

This example illustrates the SDA amplification of Factor V Leiden and the subsequent detection of the amplified nucleic acid on a NanoXhip® microarray (Nanogen, La Jolla, Calif.). Factor V Leiden (sometimes Factor V Leiden) is a hypercoagulability disorder in which Factor V, one of the coagulation factors, cannot be deactivated. Factor V Leiden is the most common hereditary hypercoagulability clotting disorder amongst Eurasians, possibly affecting up to 5% of the population of the U.S. It is named after the city Leiden (The Netherlands), where it was first identified in 1994 (Bertina et al, Nature 369: 64-67 (1994)).

Table 1. lists the oligonucleotides used in this example. The amplifiable primers (AP) contain a recognition sequence CCTCAGC (underlined) for N.BbvC1B. The bumper primer does not contain the recognition sequence. A nest and biotinylated primer (biotin-primer) was included in SDA reaction to allow post-amplification product analysis on NanoChip® platform (the biotin-primers convert the typical non-biotinylated SDA product to biotinylated product to allow anchorage of the SDA product on NanoChip® microarray).

TABLE 1

Oligonucleotide sequences

| | |
|---|---|
| Oligo name | Sequence (5′ → 3′) |

| | |
|---|---|
| Primers: | |
| FV forward AP | 5′-CATCATGAGAGACATCGCCTCCTCAGCAATAGGACTAC-3′ |
| FV reverse AP | 5′-AAATTCTCAGAATTTCTGAACCTCAGCTTCAAGGACAA-3′ |
| FV reverse bumper | 5′-GCCCCATTATTTAGCCAGGA-3′ |
| FV nest primer | 5′-bio-TGTAAGAGCAGATCCCTGGAC-3′ |
| Reporters: | |
| FV Wt disc | 5′-CTGAGTCCGAACATTGAGTCCTGTATTCCTCG-3′ |
| FV Mut disc | 5′-GCAGTATATCGCTTGACATCCTGTATTCCTTG-3′ |
| FV stab | 5′CCTGTCCAGGGATCTGCTCTTAC 3′ |
| WT univ rep probe | 5′-CTCAATGTTCGGACTCAG-A532 |
| MUT univ rep probe | 5′-TGTCAAGCGATATACTGC-A647 |

The underlined sequence indicates the nicking recognition sequence; bio is biotin, A532 (green dye) and A647 (red dye) are Alexa Fluors (Invitrogen, Eugene, OR);

SDA of Factor V Human (FV) gDNA

Sample amplification was carried out in a 10 μL volume SDA reaction that contained 50 ng of gDNA (extracted from human whole blood), 250 nM forward and reverse amplifiable primer, 25 nM reverse bumper and 500 nM biotin-primer, 3.75 mM MgCl₂, 36 mM K₂HPO₄, pH7.6, 0.25 mM each dNTPs (dATP, dGTP, dCTP and dTTP), 4U N. BbvC1B and 4U Bst DNA polymerase diluted in Diluent A (New England Biolabs, Beverly, Mass., USA). All components, including the 2 enzymes, were added together to a 200-μL microcentrifuge tube, either on ice or at room temperature. After a gentle vortex and spin of the reaction mix, the tube was placed on a thermal cycler or a heat block set at 50° C. and allowed for 30 min incubation.

### Detection of SDA Amplified Factor V DNA on a NanoChip® Microarray

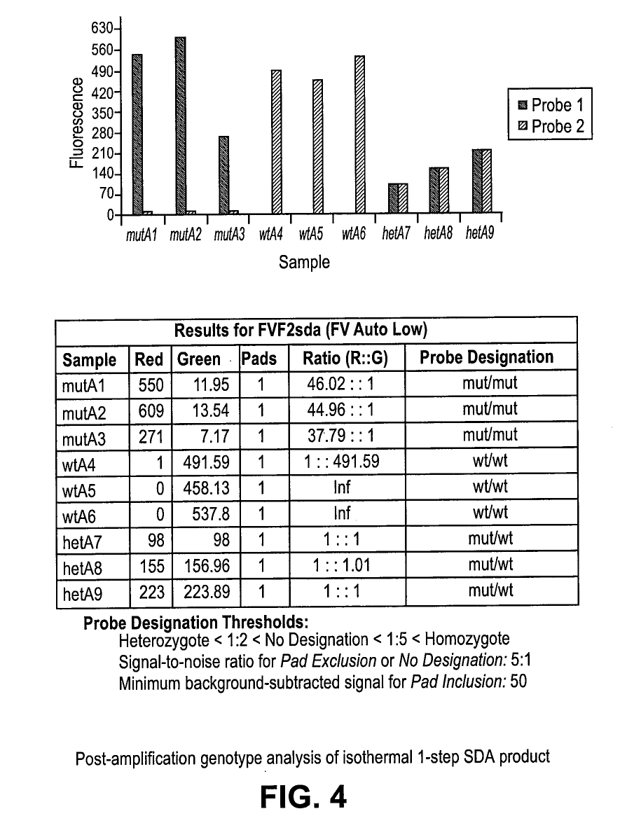

Analysis of the post-amplification product was carried out on a NanoChip® microarray. After 30 min incubation, one microliter of the SDA reaction was added to 59 μL 50 mM histidine (60 folds dilution) and electronically addressed on the Nanogen Molecular Biology WorkStation (MBW) Loader to a NanoChip® electronic microarray (Nanogen, La Jolla, Calif.) where the biotin-products in the SDA reaction would attach to the streptavidin molecules embedded in the permeation layer on the microarray while non-biotin products (including complementary strands) would be washed off the microarray. The microarray was then incubated with a reporter mix containing a stabilizer oligonucleotide, 2 discriminator oligonucleotides and 2 fluorescence labeled oligonucleotide probes for the FV wild type and mutant SNP products, respectively, and scanned on a MBW Reader. The fluorescent signal level detected on the microarray represents the yield of target product (specific to each probe) while the ratio of the 2 fluorescent signals determines the genotype of the gDNA sample. A green:red ratio >5:1 indicates a wild type sample, a green:red ratio <5:1 is for homologous mutant sample while a ratio of ˜1:1 is for heterogyzous sample. FIG. 4 shows analysis result of 9 gDNA samples that were amplified by the 1-step SDA and analyzed on a NanoChip® microarray.

### Example 3

This example illustrates the real-time SDA amplification of Factor V Leiden from human DNA detecting the amplified target with a mutant probe (5′-MGB-Q-CatAaGGAACGGA-FAM-3′) and a wild-type probe 5′-MGB-Q-CatAaGGAGCGGA-TET-3′ where MGB is the minor groove binder ligand, Q is Eclipse Dark Quencher, FAM is fluorescein, TET is tetrachloro-6-carboxyfluorescein (Glen Research, Stirling, Va.), “a” and “t” are Super A and Super T; and the bold and underlined letter indicates the SNP base.

Real Time 1-Step SDA for Human gDNA Genotype Analysis

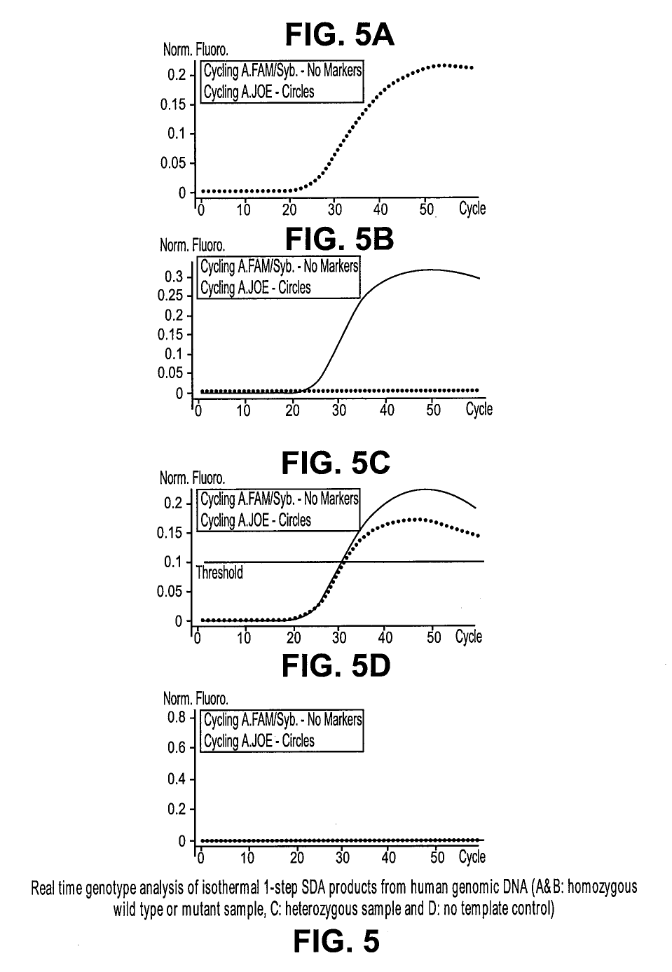

Real time genotyping analysis was successfully incorporated into the 1-step SDA. The reaction was run in 0.1 mL Strip Tubes (Corbett Robotics, Australia) in a 10 L volume reaction that had a similar composition to the above SDA reaction (but did not contain the nest biotin-primer) and contained 1× dilution of the MGB Eclipse probe mix for the human FV SNP (the probes were designed and manufactured by Nanogen Bothell and the real time probe mix contains two labeled probes, one for FV wild type and one for FV mutant product, both incorporated with the Eclipse™ Dark Quencher, the MGB™ technology and modified Super bases). The reactions, prepared at room temperature, were incubated on a Rotor-Gene 3000™ Four-Channel Multiplexing System (Corbett Robotics, Australia) set at 45° C. and fluorescent signals were collected every 20 seconds during incubation. FIG. 5 shows the real time fluorescent signals from 4 SDA reactions each contained a wild type, a mutant, a heterogyzous gDNA, respectively (one no template control). All real time signals were analyzed by the RG-3000™ software for allele discrimination.

One of ordinary skill in the art will recognize from the provided description, figures, and examples, that modifications and changes can be made to the various embodiments of the invention without departing from the scope of the invention defined by the following claims and their equivalents. Additionally, all references, patents, patent publications and the like are expressly incorporated herein by reference in their entirety for all purposes.

What is claimed is:

1. A method for generating a target nucleic acid sequence for amplification, said method comprising:
(a) providing a double stranded target sequence;
(b) nicking one strand of said target sequence with a nicking enzyme,
thereby generating the target nucleic acid sequence without thermal denaturation of the double stranded target sequence, wherein the recognition site of the nicking enzyme:
(i) is at least 6 nucleotides in length,
(ii) is present in one strand of the target sequence about 1 to about 50 times, or
(iii) comprises a combination of (i) and (ii).

2. The method of claim 1, wherein the recognition site of the nicking enzyme is at least 6 nucleotides in length.

3. The method of claim 1, wherein the recognition site of the nicking enzyme is about 7 to about 14 nucleotides in length.

4. The method of claim 3, wherein the recognition site of the nicking enzyme is at least 7 nucleotides in length.

5. The method of claim 1, wherein the recognition site of the nicking enzyme is present in one strand of the target sequence about 1 to about 50.

6. The method of claim 5, wherein the recognition site of the nicking enzyme is present in one strand of the target sequence about 9 times.

7. The method of claim 1, wherein the recognition site of the nicking enzyme is at least 7 nucleotides in length and is present in one strand of the target sequence about 9 times.

8. The method of claim 1, wherein the nicking enzyme is a type IIS nicking enzyme.

9. The method of claim 8, wherein the nicking enzyme is a modified type IIS nicking enzyme.

10. The method of claim 8, wherein the nicking enzyme is a member selected from the group consisting of: Nt.BbvCI, Nb.BsmI, N. BbvC IA, N.BbvC IB, N.BstNB I, N.Alw I, Nb.Bpu101, N.Bst9I, NMlyI, R.BbvCI, Nb.SapI-1 (variant 33) and Nb.SapI-1 (E250K).

11. The method of claim 8, wherein the nicking enzyme is Nt.BbvCI.

12. A method for amplifying a target nucleic acid sequence, said method comprising:
(a) generating a target nucleic acid sequence according to the method of claim 1;
(b) contacting a first extension primer and a first bumper primer with the target nucleic acid sequence under conditions sufficient to allow first extension primer to hybridize to the target nucleic acid sequence and for the first bumper primer to hybridize to the target nucleic sequence at a site 5′ to the binding site of the first extension primer,
wherein the 3′ end of the first extension primer comprises a target binding sequence and the 5′ end of the first extension primer comprises:
(i) a recognition sequence for the nicking enzyme and
(ii) a sequence which is complementary to the target nucleic acid,
(c) simultaneously extending the first extension primer and the first bumper primer with a polymerase to produce a first extension product and a first bumper extension product that displaces the first extension product;
(d) contacting a second extension primer and a second bumper primer with the displaced first extension product under conditions sufficient to allow the second extension primer to hybridize to the first extension product and for the second bumper primer to hybridize to the first extension product at a site 5′ to the binding site of the second extension primer,
wherein the 3′ end of the second extension primer comprises a sequence that binds to the first extension product and the 5′ end of the second extension primer comprises:
(i) a recognition sequence for the nicking enzyme and
(ii) a sequence which is complementary to the target nucleic acid; and
(e) simultaneously extending the second extension primer and the second bumper primer with the polymerase to produce a second extension product and a second bumper extension product that displaces the second extension product, thereby generating an amplified target sequence.

13. The method of claim 12, wherein the polymerase is a DNA polymerase without 5′→3′ exonuclease activity.

14. The method of claim 13, wherein the polymerase is a member selected from the group consisting of: Bst DNA Polymerase Large Fragment, Bca DNA polymerase, Klenow fragment of DNA polymerase I, Phi29 DNA polymerases, Sequenase 2.0 T7 DNA Polymerase and T5 DNA polymerase.

15. The method of claim 12, wherein the recognition site of the nicking enzyme is at least 7 nucleotides in length and is present in one strand of the target sequence about 9 times.

16. The method of claim 12, wherein the nicking enzyme is a type IIS nicking enzyme.

17. The method of claim 12, wherein the nicking enzyme is a modified type IIS nicking enzyme.

18. The method of claim 16, wherein the nicking enzyme is N.BbvC I.

19. The method of claim 12, further comprising:
(f) contacting the first extension primer to the second extension product under conditions sufficient to allow the first extension primer to hybridize to the second extension product and extending the first extension primer with the polymerase to generate a double stranded product comprising restriction sites recognized by the nicking enzyme;
(g) contacting the double stranded product with the nicking enzyme under conditions sufficient to allow the nicking enzyme to cleave a single strand of the double stranded product, thereby generating a nicked double stranded product with a nick site on each strand;
(h) contacting the first and second extension primer with the nicked double stranded product under conditions sufficient to allow the first and second extension primers to hybridize to the nicked double stranded product; and
(i) extending the first and second extension primers with a polymerase, thereby releasing single stranded amplified target sequences into solution.

20. The method of claim 19, wherein the polymerase is a DNA polymerase without 5′→3′ exonuclease activity.

21. The method of claim 19, wherein the recognition site of the nicking enzyme is at least 7 nucleotides in length and is present in one strand of the target sequence about 9 times.

22. The method of claim 19, wherein the nicking enzyme is a type IIS nicking enzyme.

23. The method of claim 19, wherein the nicking enzyme is Nt.BbvCI.

24. The method of claim 19, further comprising:
(h) detecting the amplified target sequence.
